(19) United States
(12) Patent Application Publication
(10) Pub. No.: US 2013/0202623 A1
(43) Pub. Date: Aug. 8, 2013

(54) PD-1 MODULATION AND USES THEREOF FOR MODULATING HIV REPLICATION

(71) Applicants: Nicolas Chomont, Port St. Lucie, FL (US); Rafick-Pierre Sekaly, Port St. Lucie, FL (US); Elias Haddad, Port St. Lucie, FL (US)

(72) Inventors: Nicolas Chomont, Port St. Lucie, FL (US); Rafick-Pierre Sekaly, Port St. Lucie, FL (US); Elias Haddad, Port St. Lucie, FL (US)

(21) Appl. No.: 13/579,323

(22) PCT Filed: Feb. 16, 2011
(86) PCT No.: PCT/CA2011/050096

§ 371 Date: Dec. 28, 2012

Related U.S. Application Data
(60) Provisional application No. 61/304,864, filed on Feb. 16, 2010.

(51) Int. Cl. A61K 39/395; A61K 45/06

(52) U.S. Cl. CPC A61K 39/3955; A61K 45/06
USPC 424/173.1; 435/375

(57) ABSTRACT

Methods, uses, compositions and kits for modulating HIV replication based on PD-1 modulation are disclosed. Methods, uses, compositions and kits useful for the elimination of latent HIV reservoirs based on PD-1 inhibition are also disclosed. Methods and kits useful for identifying agents useful for modulating HIV replication are also disclosed.

### CROSS REFERENCE TO RELATED APPLICATION

This application claims the benefit of U.S. Provisional Patent Application Ser. No. 61/304,864 filed on Feb. 16, 2010, which is incorporated herein by reference in its entirety.

### TECHNICAL FIELD

The present invention generally relates to the modulation of Human Immunodeficiency Virus (HIV) infection, and more particularly to methods and compositions for inhibiting or enhancing HIV replication.

### BACKGROUND ART

Human Immunodeficiency Virus-1 (HIV-1) is the etiologic agent that is responsible for Acquired Immunodeficiency Syndrome (AIDS), a syndrome characterized by depletion of CD4⁺ T lymphocytes and collapse of the immune system. HIV-1 infection is pandemic and HIV-associated diseases have become a world-wide health problem. Upon infection, HIV integrates into the cellular genome of an infected cell. HIV-1 infection then leads to two different scenarios: productive infection and latent infection. Productive infection occurs most frequently and leads to death of the infected cell after release of progeny virus. During latent infection, which is rare, HIV genes are not expressed after proviral integration, resulting in an infected cell that is characterized by transcriptionally silent HIV genes. These fully replication-competent HIV can persist dormant in cells for several years and then become reactivated (Chun et al., 1995, Nat Med 1(12):1284-1290; Chun et al., 1997, Proc Natl Aced Sci USA 94(24):13193-13197).

Current treatments of HIV infection typically seek to block one or more steps involved in the production of viral particles. Treatment options involve administration of reverse transcriptase inhibitors, inhibitors of viral protease, fusion, entry, or integration inhibitors in different combinations to block multiple steps in the viral life cycle. This approach, termed highly active antiviral therapy (HAART) has greatly decreased morbidity and mortality in people infected with HIV (Palella et al., 1998, N Engl J Med 338(13):855-860). However, there are several concerns about HAART regimens, including serious side effects of the drugs, complexity of the regimens, requirement of lifelong adherence and development of drug resistance (particularly in cases of non-compliance).

Furthermore, studies have shown that HAART is not effective in completely eradicating HIV in patients. In most cases, a rapid rebound in viremia occurs upon discontinuation of HAART, even after several years of successful treatment with undetectable viral loads (Davey et al., 1999, Proc Natl Acad Sci USA 96(26):15109-15114; Cohen and Fauci, 2001, Adv Intern Med 46: 207-246). It is believed that this rebound in viremia is due, at least in part, to the reactivation of latent HIV that persists in a small fraction of resting memory CD4⁺ T cells. Although the frequency of latently-infected CD4⁺ T cells (typically referred to as the HIV reservoir) is very low, this latent population of HIV serves as a source of virus for reseeding the infection after HAART discontinuation.

There is thus a need for novel strategies for modulating HIV replication, and for the treatment of associated conditions such as AIDS.

The present description refers to a number of documents, the content of which is herein incorporated by reference in their entirety.

### SUMMARY OF THE INVENTION

The present invention relates to the modulation of Human Immunodeficiency Virus (HIV) infection, and more particularly to methods, compositions, uses and kits for inhibiting or enhancing HIV replication.

In a first aspect, the present invention provides a method for inhibiting Human Immunodeficiency Virus (HIV) replication in a cell comprising contacting said cell with a Programmed Death-1 (PD-1) agonist.

In another aspect, the present invention provides a use of a PD-1 agonist for inhibiting HIV replication in a cell.

In another aspect, the present invention provides a use of a PD-1 agonist for the preparation of a medicament for inhibiting HIV replication in a cell.

In another aspect, the present invention provides a use of a PD-1 inhibitor for increasing HIV replication in a cell.

In another aspect, the present invention provides a use of a PD-1 inhibitor for the preparation of a medicament for increasing HIV replication in a cell.

In another aspect, the present invention provides a method for increasing HIV replication in a cell comprising contacting said cell with a PD-1 inhibitor.

In another aspect, the present invention provides a use of a PD-1 inhibitor for increasing HIV replication in a cell.

In another aspect, the present invention provides a use of a PD-1 inhibitor for the preparation of a medicament for increasing HIV replication in a cell.

In another aspect, the present invention provides a method for reducing or eliminating a latent HIV reservoir in a cell comprising: (a) performing the method for increasing HIV replication in a cell defined above; and contacting said cell with one or more antiretroviral agents.

In another aspect, the present invention provides a method for decreasing the number of latently HIV-infected cells in a subject, said method comprising administering to said subject an effective amount of: (a) a PD-1 inhibitor; and (b) one or more antiretroviral agents.

In another aspect, the present invention provides a use of (i) a PD-1 inhibitor and (ii) one or more antiretroviral agents for eliminating a latent HIV reservoir in a cell.

In another aspect, the present invention provides a use of (i) a PD-1 inhibitor and (ii) one or more antiretroviral agents for the preparation of a medicament for eliminating a latent HIV reservoir in a cell.

In another aspect, the present invention provides a use of (i) a PD-1 inhibitor and (ii) one or more antiretroviral agents for decreasing the number of latently HIV-infected cells in a subject.

In another aspect, the present invention provides a use of (i) a PD-1 inhibitor and (ii) one or more antiretroviral agents for the preparation of a medicament for decreasing the number of latently HIV-infected cells in a subject.

In another aspect, the present invention provides a composition for inhibiting HIV replication in a cell, said composition comprising a PD-1 agonist and a carrier.

In another aspect, the present invention provides a composition for inhibiting HIV replication in an HIV-infected, said composition comprising a PD-1 agonist and a carrier.

In another aspect, the present invention provides a composition for increasing HIV replication in a cell, said composition comprising a PD-1 inhibitor and a carrier.

In another aspect, the present invention provides a composition for reducing or eliminating a latent HIV reservoir in a cell, said composition comprising a PD-1 inhibitor, one or more antiretroviral agents, and a pharmaceutically acceptable carrier.

In another aspect, the present invention provides a composition for decreasing the number of latently HIV-infected cells in a subject, said composition comprising a PD-1 inhibitor, one or more antiretroviral agents, and a carrier.

In an embodiment, the above-mentioned cell is a CD4⁺ T cell. In an embodiment, the above-mentioned agonist is a PD-1 ligand. In a further embodiment, the above-mentioned PD-1 ligand comprises a PD-L1 polypeptide or an extracellular domain thereof (having PD-1 agonist activity). In a further embodiment, the above-mentioned PD-1 ligand comprises an extracellular domain of PD-L1 (having PD-1 agonist activity) linked to an antibody Fc domain. In an embodiment, the above-mentioned antibody Fc domain is a human IgG₁ domain.

In an embodiment, the above-mentioned PD-1 inhibitor blocks the interaction between PD-1 and a PD-1 ligand. In a further embodiment, the above-mentioned PD-1 ligand is PD-L1.

In an embodiment, the above-mentioned PD-1 inhibitor is an anti-PD-1 antibody or antigen-binding fragment thereof.

In another embodiment, the above-mentioned cell is a latently HIV-infected cell. In a further embodiment, the above-mentioned latently-infected cell is a CD4⁺ T cell.

In another aspect, the present invention provides a method for determining whether a test compound may be useful for inhibiting HIV replication, said method comprising:

(a) contacting a cell expressing PD-1 or a functional variant or fragment thereof with said test compound;

(b) determining whether PD-1 activity is increased in the presence of said test compound relative to the absence thereof;

wherein an increase in said activity in the presence of said of said test compound relative to the absence thereof is indicative that said test compound may be useful for inhibiting HIV replication.

In another aspect, the present invention provides a method for determining whether a test compound may be useful for decreasing the number of latently HIV-infected cells in a subject, said method comprising:

(a) contacting a cell expressing PD-1 or a functional variant or fragment thereof with said test compound;

(b) determining whether PD-1 activity is decreased in the presence of said test compound relative to the absence thereof;

wherein a decrease in said activity in the presence of said of said test compound relative to the absence thereof is indicative that said test compound may be useful for decreasing the number of latently HIV-infected cells in a subject.

In another aspect, the present invention provides a method for determining whether a test compound may be useful for increasing HIV replication in a cell, said method comprising:

(a) contacting a cell expressing PD-1 or a functional variant or fragment thereof with said test compound;

(b) determining whether PD-1 activity is decreased in the presence of said test compound relative to the absence thereof;

wherein a decrease in said activity in the presence of said of said test compound relative to the absence thereof is indicative that said test compound may be useful for increasing HIV replication in a cell.

In another aspect, the present invention provides a method for obtaining a cell population enriched in latently HIV-infected cells, the method comprising: contacting said cell population with an agent binding to PD-1; and isolating/purifying the cells on which the ligand is bound, thereby obtaining a cell population enriched in latently HIV-infected cells.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIGS. 1A-1C show the frequency of CD4⁺ T cells expressing PD-1 in HIV-infected subjects. FIG. 1A: Correlation between the frequency of CD4⁺ T cells expressing PD-1 and the frequency of CD4⁺ T cells harbouring HIV integrated DNA in a cohort of 32 HIV-infected subjects receiving suppressive HAART. FIG. 1B: Frequency of CD4⁺ T cells expressing PD-1 in HIV negative controls (circles; n=8), HIV-infected subjects receiving suppressive HAART (squares; n=9) and HIV-infected untreated subjects (triangles; n=10). FIG. 1C: Frequency of naïve (CD45RA⁺ CCR7⁺ CD27⁺, T_{N}), central memory (CD45RA⁻ CCR7⁺ CD27⁺, T_{CM}), transitional memory (CD45RA⁻ CCR7⁻ CD27⁺, T_{TM}) and effector memory (CD45RA⁻ CCR7⁻ CD27⁻, T_{EM}) CD4⁺ T cells expressing PD-1 measured in CD4⁺ T cells from 9 virally suppressed subjects. PD-1 expression was measured by flow cytometry in total CD4⁺ T cells (FIGS. 1A and 1B) or in gated memory CD4 T cells subsets using the CD45RA, CCR7, and CD27 markers (FIG. 1C).

FIGS. 2A and 2B shows the frequency of PD-1^{hi} (left bar of each pair) and PD-1^{lo} (right bar of each pair) cells harbouring HIV DNA and integrated HIV DNA in untreated HIV-infected subjects (FIG. 2A) and virally-suppressed subjects (FIG. 2B). Memory CD4⁺ T cell subsets (T_{CM}, T_{TM} and T_{EM}) from 2 untreated, viremic subjects and 2 HAART-treated, virally-suppressed subjects were sorted according to their relative expression of PD-1. Sorted cells were subjected to ultrasensitive quantitative PCR to measure the frequency of cells harbouring HIV DNA and integrated HIV DNA.

FIG. 3 shows the effect of PD-1 triggering on HIV replication in primary CD4⁺ T cells. CD4⁺ T cells from 4 viremic donors were isolated by magnetic negative selection and activated with beads coated with anti-CD3+anti-CD28 antibodies and with the Fc-PD-L1 chimera, or the appropriate isotype (IgG2) control (NS=non stimulated). Cell supernatants were collected after 3 (d3), 6 (d6) and 9 (d9) days of culture, and viral replication was measured by p24 ELISA;

FIGS. 4A-4C show the effect of PD-1 triggering on early HIV replication in primary CD4⁺ T cells. CD4⁺ T cells from 7 viremic donors were isolated by negative selection and activated with beads coated with anti-CD3+anti-CD28 antibodies and with the Fc-PD-L1 chimera, or the appropriate isotype (IgG2) control. Cell supernatants were collected after 24 hours of stimulation, and viral particles were pelleted by ultracentrifugation. After extraction of viral RNA, viral production was measured by ultrasensitive real time RT-PCR. FIGS. 4A and 4B show the raw data obtained in five representative donors, and FIG. 4C shows the mean values and standard deviations (SD) obtained from 7 independent experiments, expressed as a percentage of viral production relative to the positive control (anti-CD3+anti-CD28 antibodies and isotype (IgG2) control).

FIGS. 5A and 5B show the effect of PD-1 triggering on “early” (24 h, FIG. 5A) or “late” (3, 6 and 9 days, FIG. 5B) HIV replication in primary CD4⁺ T cells in the presence of antiretroviral molecules (ARV). CD4⁺ T cells from 6 viremic donors were isolated by negative selection and activated with beads coated with anti-CD3+anti-CD28 antibodies and with the Fc-PD-L1 chimera, or the appropriate isotype (IgG2) control, in the presence of antiretroviral molecules (ARV). FIG. 5A: Cell supernatants were collected after 24 hours of stimulation, and viral particles were pelleted by ultracentrifugation. After extraction of viral RNA, viral production was measured by ultrasensitive real time RT-PCR. The data obtained in 4 representative subjects are depicted. FIG. 5B: Cell supernatants were collected after 3 (d3), 6 (d6) and 9 (d9) days of culture, and viral replication was measured by p24 ELISA. Circles: non-stimulated (NS)+ARV; triangles: anti-CD3+anti-CD28 antibodies+Fc-PD-L1 chimera; squares: anti-CD3+anti-CD28 antibodies+isotype (IgG2) control. The data obtained in 2 representative subjects are depicted;

FIG. 6 shows the effect of PD-1 triggering in primary CD4⁺ T cells expressing high (top panel) or low (bottom panel) levels of PD-1. Memory CD4 T cells (CD3⁺ CD4⁺ CD45RA⁻) from 2 untreated subjects were sorted according to their relative expression of PD-1 and activated with beads coated with anti-CD3+anti-CD28 antibodies and with the Fc-PD-L1 chimera, or the appropriate isotype (IgG2) control. Cell supernatants were collected after 3 days of culture, and viral replication was measured by p24 ELISA;

FIG. 7 shows the effect of blocking the PD-1/PD-L1 interaction on viral production in CD4⁺ T cells. CD4⁺ T cells from 3 viremic donors were isolated by negative selection and incubated with a monoclonal anti-PD-1 antibody (ONO-4538), a fully human IgG4 (Medarex Inc.; Cat. No. MDX-1106). The anti-PD-1 human monoclonal antibody MDX-1106 binds to PD-1 and prevents the interaction with its ligands PD-L1 and PD-L2. Cell supernatants were collected after 3 days and viral replication was measured by p24 ELISA;

FIGS. 8A and 8B show the amino acid (SEQ ID NO: 2) and nucleotide (SEQ ID NO: 1) sequences, respectively, of human PD-1. The signal peptide is indicated in italics in the amino acid sequence, and the coding region is indicated in bold in the nucleotide sequence;

FIGS. 9A and 9B show the amino acid (SEQ ID NO: 4) and nucleotide (SEQ ID NO: 3) sequences, respectively, of human PD-L1. The signal peptide is indicated in italics in the amino acid sequence, and the coding region is indicated in bold in the nucleotide sequence;

FIGS. 10A and 10B show the amino acid (SEQ ID NO: 14) and nucleotide (SEQ ID NO: 13) sequences, respectively, of human PD-L2. The signal peptide is indicated in italics in the amino acid sequence, and the coding region is indicated in bold in the nucleotide sequence;

FIG. 11 shows an amino sequence alignment of mouse and human PD-L1 and PD-L2 (from Latchman et al., 2001, Nature Immunology 2: 261-268);

FIG. 12 shows an amino sequence alignment of the ectodomains of mouse and human PD-1.

### DISCLOSURE OF INVENTION

### Inhibition of HIV Replication

In the studies described herein, the present inventors have shown that modulating PD-1 activity has an effect on HIV replication in primary CD4⁺ T cells obtained from chronic HIV-infected subjects. More specifically, they have shown that engagement of PD-1 using its natural ligand PD-L1 results in an inhibition of HIV replication in activated primary CD4⁺ T cells from HIV-infected subjects.

Accordingly, in a first aspect, the present invention provides a method for inhibiting HIV replication in a cell comprising contacting said cell with a PD-1 agonist. The present invention also provides a method for treating HIV infection, as well as treating a related condition such as AIDS, in a subject, comprising administering to said subject an effective amount of a PD-1 agonist. The present invention also provides a use of a PD-1 agonist for inhibiting HIV replication in a cell, or for the preparation of a medicament for inhibiting HIV replication in a cell. The present invention also provides a use of a PD-1 agonist for treating HIV infection in a subject (as well as treating a related condition such as AIDS), or for the preparation of a medicament for treating HIV infection in a subject (as well as treating a related condition such as AIDS). The present invention also provides a composition for inhibiting HIV replication in a cell and/or for treating HIV infection in a subject (as well as treating a related condition such as AIDS), said composition comprising a PD-1 agonist and a pharmaceutically acceptable carrier or excipient. In an embodiment, the above-mentioned cell is a latently HIV-infected cell. In another embodiment, the above-mentioned cell is a CD4⁺ T cell, in a further embodiment a memory CD4⁺ T cell, in a further embodiment a particular subset of memory CD4⁺ T cell, such as a central memory (CD45RA⁻ CCR7⁺ CD27⁺, T_{CM}), transitional memory (CD45RA⁻ CCR7⁻ CD27⁺, T_{TM}) or effector memory (CD45RA⁻ CCR7⁻ CD27⁻, T_{EM}) CD4⁺ T cell. In another embodiment, the above-mentioned cell expresses PD-1.

PD-1, a member of the immunoglobulin (Ig) superfamily, is highly upregulated on activated lymphocytes and monocytes. It interacts with its two known ligands PD-L1 (B7-H1) and PD-L2 (B7-DC). PD-L1 is constitutively expressed on splenic T cells, B cells, monocytes, macrophages and dendritic cells (DCs), and its expression can be induced by activation of T lymphocytes, monocytes, macrophages and DCs. PD-L2 is expressed on non-lymphoid tissues and is upregulated on monocytes and DCs after activation.

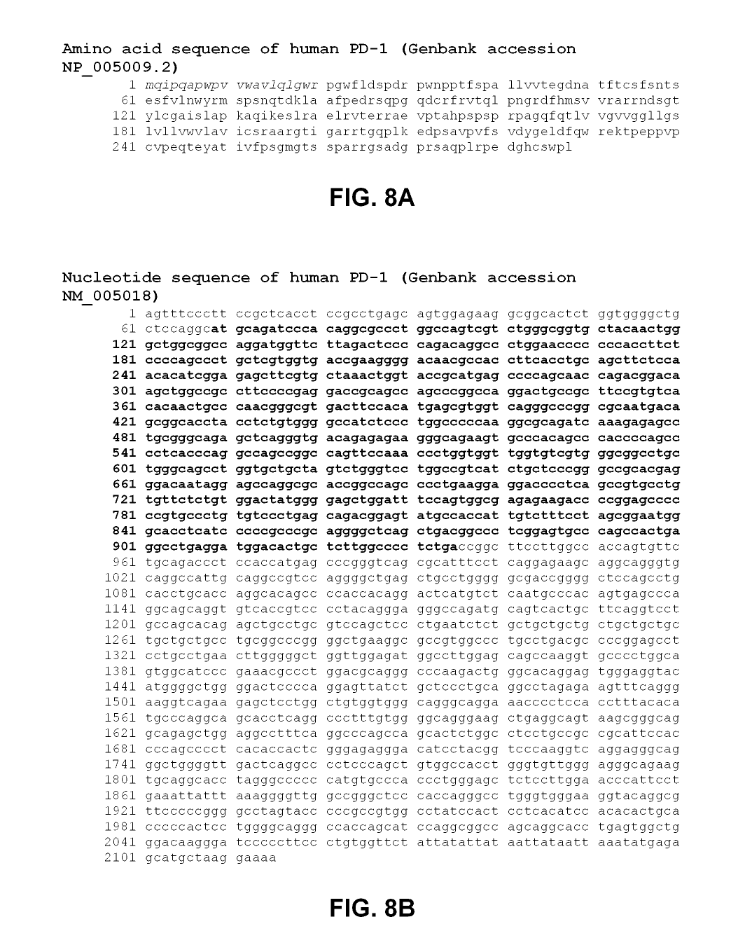

Human PD-1 is a Type I membrane protein of 268 amino acids (precursor=288 amino acids) comprising an extracellular portion (from about residues 21 to 170) that includes an IgV domain (from about residues 35 to 145), a transmembrane domain (from about residues 171 to 191 and an intracellular tail (from about residues 192 to 288). The intracellular tail contains two phosphorylation sites located in an immunoreceptor tyrosine-based inhibitory motif (residues 221 to 226) and an immunoreceptor tyrosine-based switch motif (residues 246 to 251). These two motifs are involved in the recruitment of the phosphatases SHP-1 and SHP-2, which at least in part mediates the inhibitory activity of PD-1 (Sheppard et al., 2004, FEBS Letters, 574(1-3): 37-41). The amino acid and nucleotide sequences of human PD-1 are shown in FIGS. 8A and 8B, respectively.

“PD-1 agonist” as used herein refers to any agent capable of inducing/triggering the PD-1 signalling pathway in a cell. It includes agents that binds to PD-1 (e.g., to the extracellular portion of PD-1) and triggers an intracellular signal, such as a natural or synthetic PD-1 ligand (e.g., an agonistic antibody, as described in PCT publications Nos. WO 04/056875, WO 10/029,434 and WO 10/029,435). In an embodiment, the above-mentioned PD-1 agonist is a natural PD-1 ligand (e.g., PD-L1, PD-L2), or a functional variant or fragment thereof (a variant or fragment exhibiting PD-L1 or PD-L2 activity). In a further embodiment, the above-mentioned natural PD-1 ligand is PD-L1 or a functional variant or fragment thereof.

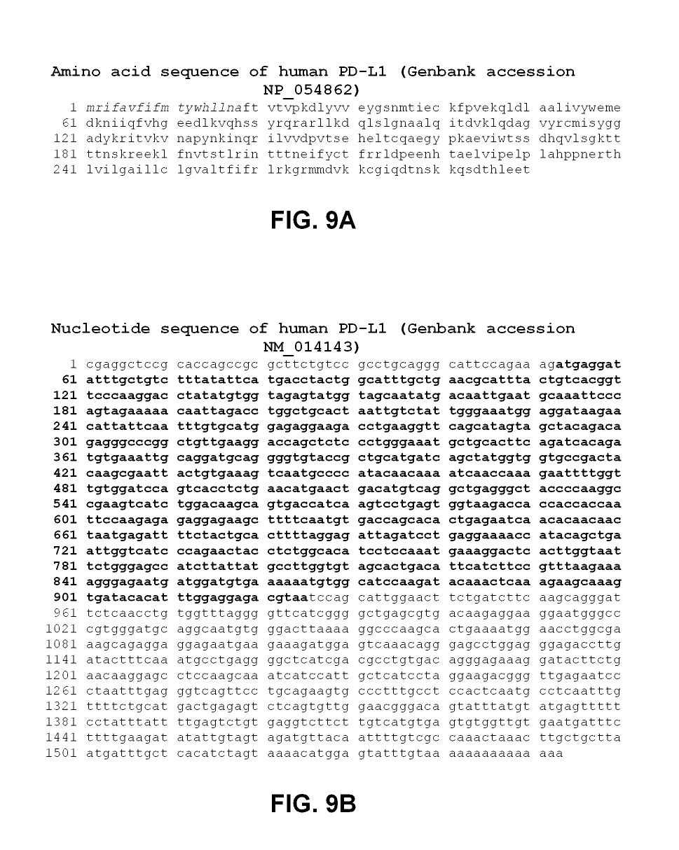

Human PD-L1 is a Type I membrane protein of 272 amino acids (precursor=290 amino acids) comprising an extracellular portion (from about residues 19 to 238) that includes an IgV domain (from about residues 18 to 130), a transmembrane domain (from about residues 239 to 261 and a short intracellular tail (from about residues 262 to 290). The amino acid and nucleotide sequences of human PD-L1 are shown in FIGS. 9A and 9B, respectively. Human

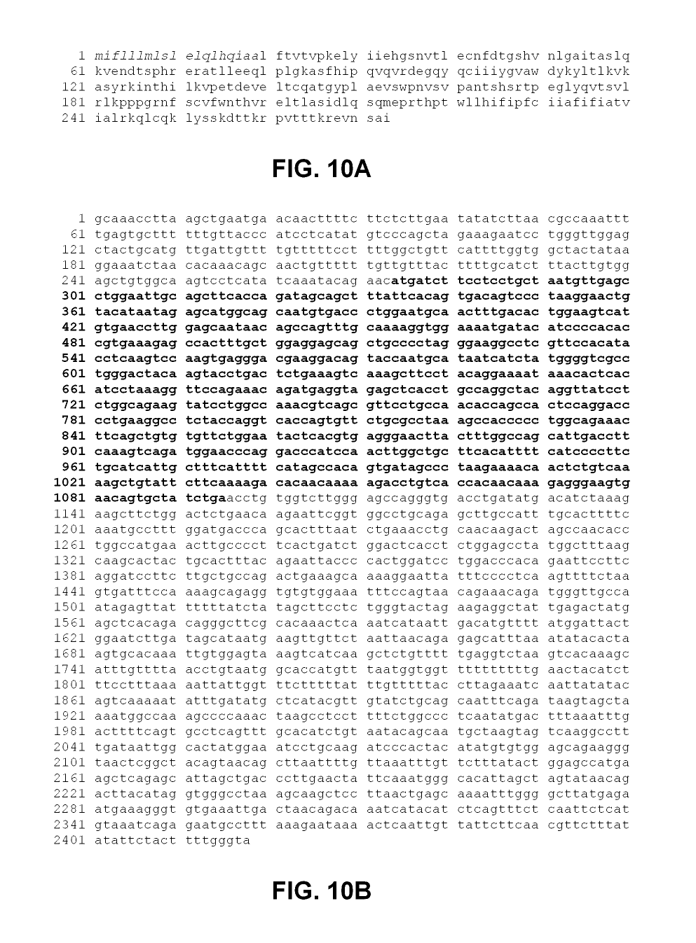

PD-L2 is a Type I membrane protein of 253 amino acids (precursor=273 amino acids) comprising an extracellular portion of about 201 amino acids that includes an IgV domain (about residues 35 to 120) and a C-like Ig domain (about residues 137 to 193), a transmembrane domain of about 24 residues (about residues 221-241) and a short intracellular tail of about 28 residues. The amino acid and nucleotide sequences of human PD-L2 are shown in FIGS. 10A and 10B, respectively.

Functional variants and fragments of PD-L1 or PD-L2 as used herein refers to variants (PD-L1/PD-L2 mutants having one or more substitutions, deletions and/or additions relative to native PD-L1/PD-L2) or fragments of PD-L1/PD-L2 (e.g., the extracellular portion of PD-L1/PD-L2), which retain the activity of native PD-L1/PD-L2, such as the ability to bind PD-1 and to trigger a signal through PD-1. In an embodiment, the above-mentioned PD-1 agonist comprises a fragment of PD-L1/PD-L2, such as the extracellular fragment of PD-L1/PD-L2. In a further embodiment, the above-mentioned PD-L1/PD-L2 fragment comprises the IgV domain. In another embodiment, the above-mentioned PD-L1 fragment comprises one or more of residues 19, 20, 26, 54, 56, 66, 113, 115, 117, and 121-125 of the IgV domain of PD-L1.

In an embodiment, the above-mentioned PD-L1/PD-L2 derivative is a PD-L1/PD-L2, or a fragment thereof (e.g., the extracellular fragment of PD-L1/PD-L2), linked to an Fc portion of an antibody (directly or via a linker), such as the Recombinant Human B7H1/PD-L1 Fc Chimera commercially available from R&D Systems™ (Cat. No. 156-B7), which comprises residues Phe19 to Thr167 of human PD-L1 linked to residues Pro100 to Lys330 of human IgG₁ via a linker (sequence: DIEGRMD, SEQ ID NO: 11), or the Recombinant Human PD-L2 Fc Chimera commercially available from R&D Systems™ (Cat. No. 1224-PL), which comprises residues Leu20 to Pro219 of human PD-L2 linked to residues Pro100 to Lys330 of human IgG₁ via a linker (sequence: IEGRMD, SEQ ID NO: 12).

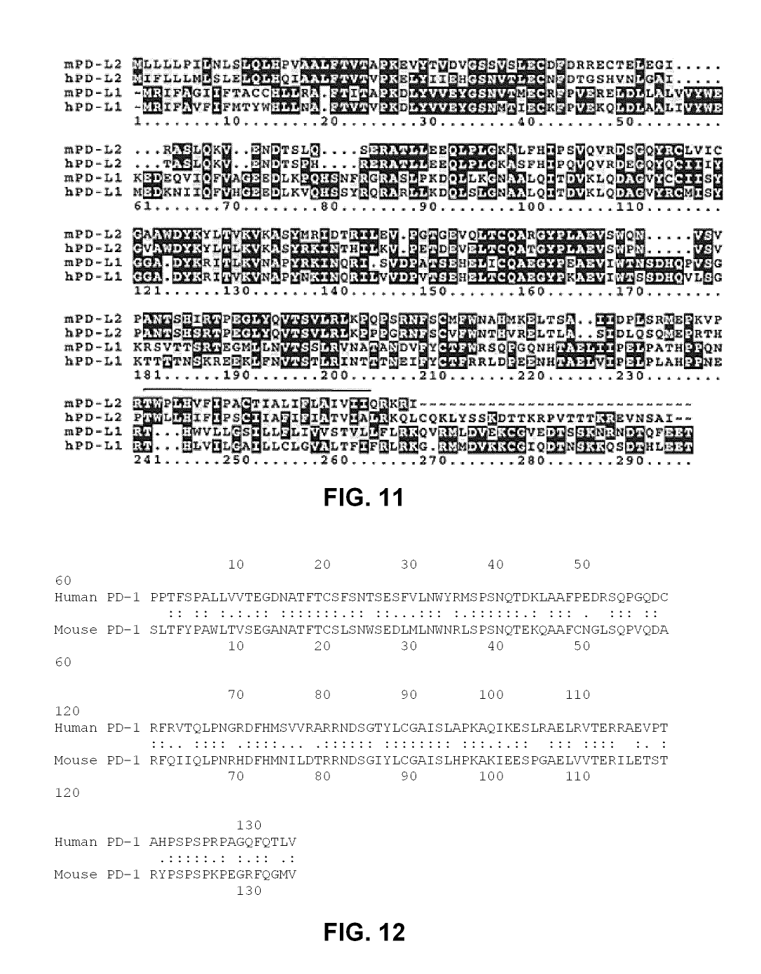

The domains and residues of human PD-1 and PD-L1 involved in their interaction is described in for example Lin et al., Proc. Natl. Acad. Sci. 2008 105(8): 3011-3016. The IgV domains of PD-1 (from about residues 35 to 145, and more particularly residues 64, 66, 68, 73-76, 78, 90, 122, 124, 126, 128, 130-132, 134 and 136) and PD-L1 (from about residues 18 to 130, and more particularly residues 19, 20, 26, 54, 56, 66, 113, 115, 117, and 121-125) are involved in the interaction. Similarly, the domains and residues of mouse PD-1 and PD-L1 involved in their interaction is described in for example Lazar-Molnar et al., Proc. Natl. Acad. Sci. 2008 105(30): 10483-10488. The IgV domains of murine PD-1 (more particularly residues 31, 33, 35, 40, 42, 43, 45, 50, 95, 99 and 103) and murine PD-L2 (more particularly residues 21, 28, 56, 60, 101, 110, 112, 113 and 114) are involved in the interaction. It may be expected that the most or all corresponding residues of human PD-1 and PD-L2 (which may be readily identified by sequence comparison/alignment, FIGS. 11 and 12) also interact with each others (Lin et al., 2008, supra; Lazar-Molnar et al., 2008, supra). Based on this knowledge, the skilled person would be able to identify/prepare active (which may be used as agonists) and/or inactive (which may be used as PD-1 inhibitors) fragments and/or variants of PD-1/PD-L1/PD-L2, as well as compounds/agents (e.g., peptides, antibodies, small molecules) capable of blocking the PD-1-PD-L1/PD-L2 interaction.

As used herein, the terms “treat”, “treating”, and “treatment” include inhibiting the condition or disease, i.e., arresting or reducing the development or progression of the condition or disease or its clinical symptoms; or relieving the condition or disease, i.e. causing regression of the condition or disease or its clinical symptoms. Treatment means any manner in which the symptoms or pathology of a condition, disorder, or disease are ameliorated or otherwise beneficially altered.

In further embodiments, the methods of the invention are for preventing a condition or disease, i.e., causing the clinical symptoms of the condition or disease not to develop in a subject that may be predisposed to the condition or disease but does not yet experience any symptoms of the condition or disease, or reducing the onset of the condition or disease, or symptoms thereof (or severity thereof). Prevention encompasses prophylaxis.

Preferably, the subject in need of such treatment or prevention is a mammal, more preferable a human.

### Increase of HIV Replication/Reactivation of the Latent HIV Reservoir

The present inventors have further shown that an increase in HIV replication was observed following incubation of primary CD4⁺ T cells with an antibody blocking the interaction between PD-1 and PD-L1. Accordingly, in another aspect, the present invention provides a method for increasing HIV replication in a cell comprising contacting said cell with a PD-1 inhibitor. The present invention also provides a use of a PD-1 inhibitor for increasing HIV replication in a cell, or for the preparation of a medicament for increasing HIV replication in a cell.

The present invention also provides a method for reactivating HIV replication in a latently HIV-infected cell, said method comprising contacting said cell with a PD-1 inhibitor. The present invention also provides a use of a PD-1 inhibitor for reactivating HIV replication in a latently HIV-infected cell, or for the preparation of a medicament for reactivating HIV replication in a latently HIV-infected cell.

As used herein, the term “PD-1 inhibitor” includes any compound able to directly or indirectly affect the regulation of PD-1 by reducing for example the expression of PD-1 (i.e., transcription and/or the translation) or its natural ligands PD-L1/PD-L2, or a PD-1 activity. It includes intracellular (e.g., agents that block a PD-1-associated signalling molecule or pathway, such as SHP-1 and SHP-2) as well as extracellular PD-1 inhibitors. Without being so limited, such inhibitors include siRNA, antisense molecules, proteins, peptides, small molecules, antibodies, etc.

In an embodiment, the above-mentioned PD-1 inhibitor blocks/inhibits the interaction between PD-1 and a PD-1 ligand (e.g., PD-L1, PD-L2). Such inhibitor may target, for example, the IgV domain of PD-1 and/or PD-L1 and/or PD-L2, such as one or more of the residues involved in the interaction, as discussed above.

In an embodiment, the above-mentioned PD-1 inhibitor is a blocking antibody, such as an anti-PD-1 or anti-PD-L1/PD-L2 antibody. Blocking anti-PD-1 and/or anti-PD-L1/PD-L2 antibodies are well known in the art and are described, for example, in Goldberg et al., Blood 110(1): 186-192 (2007), Thompson et al., Clin. Cancer Res. 13(6): 1757-1761 (2007), Chen Y et al., Hybridoma (Larchmt) 29(2):153-60 2010); U.S. Patent Application Publication Nos. US 2003/0039653, US 2004/0213795, US 2006/0110383, US 2007/0065427 and US 2007/0122378 as well as in PCT publication Nos. WO 04/056875, WO 06/121168, WO 08/156,712, WO 09/114,335, WO 10/036,959 and WO 10/089,411, as well as antibody MDX-1106 (ONO-4538) tested in clinical studies for the treatment of certain malignancies (Brahmer et al., J Clin Oncol. 2010 28(19): 3167-75, Epub 2010 Jun. 1). Other blocking antibodies may be readily identified and prepared by the skilled person based on the known domain of interaction between PD-1 and PD-L1/PD-L2, as discussed above. For example, a peptide corresponding to the IgV region of PD-1 or PD-L1/PD-L2 (or to a portion of this region) could be used as an antigen to develop blocking antibodies using methods well known in the art.

By “anti-PD-1 antibody” or “anti-PD-L1” or “anti-PD-L2” in the present context is meant an antibody capable of detecting/recognizing (i.e. binding to) a PD-1, PD-L1 or PD-L2 protein or a PD-1, PD-L1 or PD-L2 protein fragment. In an embodiment, the above-mentioned antibody inhibits the biological activity of PD-1, such as PD-1-PD-L1/PD-L2 interaction or PD-1-mediated T cell inhibition. In another embodiment, the PD-1 or PD-L1/PD-L2 protein fragment is an extracellular domain of PD-1 or PD-L1/PD-L2 (e.g., the IgV domain).

In an embodiment, the antibody specifically binds to (interacts with) a polypeptide (e.g., the polypeptide of SEQ ID NO: 2, 4 or 14) and displays no substantial binding to other naturally occurring proteins other than the ones sharing the same antigenic determinants as a PD-1 or PD-L1/PD-L2 polypeptide. The term antibody or immunoglobulin is used in the broadest sense, and covers monoclonal antibodies (including full-length monoclonal antibodies), polyclonal antibodies, multispecific antibodies, and antibody fragments so long as they exhibit the desired biological activity. Antibody fragments comprise a portion of a full length antibody, generally an antigen binding or variable region thereof. Examples of antibody fragments include Fab, Fab′, F(ab′)2, and Fv fragments, diabodies, linear antibodies, single-chain antibody molecules, single domain antibodies (e.g., from camelids), shark NAR single domain antibodies, and multispecific antibodies formed from antibody fragments. Antibody fragments can also refer to binding moieties comprising CDRs or antigen binding domains including, but not limited to, VH regions (VH, VH-VH), anticalins, PepBodies, antibody-T-cell epitope fusions (Troybodies) or Peptibodies. Additionally, any secondary antibodies, either monoclonal or polyclonal, directed to the first antibodies would also be included within the scope of this invention.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody A Laboratory Manual, CSH Laboratories). The term antibody encompasses herein polyclonal, monoclonal antibodies and antibody variants such as single-chain antibodies, humanized antibodies, chimeric antibodies and immunologically active fragments of antibodies (e.g., Fab and Fab′ fragments) which inhibit or neutralize their respective interaction domains and/or are specific thereto. In an embodiment, the antibody is a monoclonal antibody.

Polyclonal antibodies are preferably raised in animals by multiple subcutaneous (s.c.), intravenous (i.v.) or intraperitoneal (i.p.) injections of the relevant antigen (e.g., PD-1 or PD-L1/PD-L2 polypeptide or a fragment thereof) with or without an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals may be immunized against the antigen (e.g., PD-1 or PD-L1/PD-L2 polypeptide or a fragment thereof, such as the IgV domain or a fragment thereof), immunogenic conjugates, or derivatives by combining the antigen or conjugate (e.g., 100 μg for rabbits or 5 μg for mice) with 3 volumes of Freund's complete adjuvant and injecting the solution intradermally at multiple sites. One month later the animals are boosted with the antigen or conjugate (e.g., with ⅕ to 1/10 of the original amount used to immunize) in Freund's complete adjuvant by subcutaneous injection at multiple sites. Seven to 14 days later the animals are bled and the serum is assayed for antibody titer. Animals are boosted until the titer plateaus. Preferably, for conjugate immunizations, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies may be made using the hybridoma method first described by Kohler et al., Nature, 256: 495 (1975), or may be made by recombinant DNA methods (e.g., U.S. Pat. No. 6,204,023). Monoclonal antibodies may also be made using the techniques described in U.S. Pat. Nos. 6,025,155 and 6,077,677 as well as U.S. Patent Application Publication Nos. 2002/0160970 and 2003/0083293.

In the hybridoma method, a mouse or other appropriate host animal, such as a rat, hamster or monkey, is immunized (e.g., as hereinabove described) to elicit lymphocytes that produce or are capable of producing antibodies that will specifically bind to the antigen used for immunization. Alternatively, lymphocytes may be immunized in vitro. Lymphocytes then are fused with myeloma cells using a suitable fusing agent, such as polyethylene glycol, to form a hybridoma cell.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

In an embodiment, the above-mentioned antibody is raised against an extracellular domain of a PD-1 or PD-L1/PD-L2 polypeptide (i.e. an extracellular domain of a PD-1 or PD-L1/PD-L2 polypeptide is used for immunization). In a further embodiment, the above-mentioned antibody is raised against a PD-1 or PD-L1/PD-L2 polypeptide fragment comprised in the IgV domain of a PD-1 or PD-L1/PD-L2 polypeptide.

In an embodiment, the above-mentioned antibody blocks or interferes with PD-1-PD-L1 interaction, for example by competing for the PD-L1/PD-L2 binding domain on PD-1 (or vice-versa) or by sterically hindering the PD-L1/PD-L2 binding domain on PD-1 (or vice-versa). In another embodiment, the above-mentioned antibody binds to an epitope located in the IgV domain of a PD-1 or PD-L1/PD-L2 polypeptide.

PD-1 or PD-L1/PD-L2 inhibitors may also be in the form of non-antibody-based scaffolds, such as avimers (Avidia); DARPins (Molecular Partners); Adnectins (Adnexus), Anticalins (Pieris) and Affibodies (Affibody). The use of alternative scaffolds for protein binding is well known in the art (see, for example, Binz and Pluckthun, 2005, Curr. Opin. Biotech. 16: 1-11).

In another embodiment, the PD-1 inhibitor is a PD-L1 or PD-L2 polypeptide, especially a soluble portion of PD-L1 or PD-L2, that binds to PD-1 without triggering inhibitory signal transduction, such as those described in U.S. Pat. No. 6,803,192 and PCT publication No. WO 10/027,423.

In another embodiment, the above-mentioned PD-1 inhibitor is an antisense or RNAi-based inhibitory molecule.

As used herein “antisense molecule” is meant to refer to an oligomeric molecule, particularly an antisense oligonucleotide for use in modulating the activity or function of nucleic acid molecules encoding a PD-1 polypeptide (e.g., the polypeptide of SEQ ID NO: 2) or its ligands PD-L1 or PD-L2 (e.g., the polypeptide of SEQ ID NOs: 4 or 14), ultimately modulating the amount of PD-1 and/or PD-L1 produced in cells (e.g., immune cells, latently HIV-infected cells). This is accomplished by providing oligonucleotide molecules which specifically hybridize with one or more nucleic acids encoding PD-1 and/or PD-L1. As used herein, the term “nucleic acid encoding a PD-1 (or PD-L1) polypeptide” encompasses DNA encoding said polypeptide, RNA (including pre-mRNA and mRNA) transcribed from such DNA, and also cDNA derived from such RNA (e.g., a nucleic acid comprising the coding sequence of the nucleotide sequence set forth in SEQ ID NO: 1 or 3). The specific hybridization of an oligomeric compound with its target nucleic acid interferes with the normal function of the nucleic acid. The overall effect of such interference with target nucleic acid function is modulation of the expression of PD-1 and/or PD-L1. In the context of the present invention, “modulation” means either an increase (stimulation) or a decrease (inhibition) in the expression of a gene.

In the context of this invention, “hybridization” means hydrogen bonding between complementary nucleoside or nucleotide bases. Terms “specifically hybridizable” and “complementary” are the terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed. Such conditions may comprise, for example, 400 mM NaCl, 40 mM PIPES pH 6.4, 1 mM EDTA, at 50 to 70° C. for 12 to 16 hours, followed by washing. The skilled person will be able to determine the set of conditions most appropriate for a test of complementarity of two sequences in accordance with the ultimate application of the hybridized nucleotides.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. Examples of modified nucleotides include a 2′-O-methyl modified nucleotide, a nucleotide comprising a 5′-phosphorothioate group, a terminal nucleotide linked to a cholesteryl derivative, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate and a non-natural base comprising nucleotide.

Methods to produce antisense molecules directed against a nucleic acid are well known in the art. The antisense molecules of the invention may be synthesized in vitro or in vivo.

Reagents and kits for performing RNAi are available commercially from for example

Ambion Inc. (Austin, Tex., USA), New England Biolabs Inc. (Beverly, Mass., USA) and Invitrogen (Carlsbad, Calif., USA).

The antisense molecule may be expressed from recombinant viral vectors, such as vectors derived from adenoviruses, adeno-associated viruses, retroviruses, herpesviruses, and the like. Such vectors typically comprises a sequence encoding an antisense molecule of interest (e.g., a dsRNA specific for PD-1 and/or PD-L1) and a suitable promoter operatively linked to the antisense molecule for expressing the antisense molecule. The vector may also comprise other sequences, such as regulatory sequences, to allow, for example, expression in a specific cell/tissue/organ, or in a particular intracellular environment/compartment. Methods for generating, selecting and using viral vectors are well known in the art.

Antisense molecules (siRNA and shRNA) inhibiting the expression of human PD-1 are commercially available, for example from Origene (TG310561) and from Sigma-Aldrich (Cat. No TRCN0000083508 to TRCN0000083512, and EHU146521). Also, several providers (e.g., InvivoGen, Qiagen, Ambion, Inc.) offer custom-made antisense synthesis services. PD-1 siRNA are also described in Borkner et al., Cancer Immunol Immunother. 2010 59(8):1173-83, Epub 2010 Mar. 27. Similarly, antisense molecules (siRNA and shRNA) inhibiting the expression of human PD-L1 are commercially available, for example from Santa Cruz Biotechnology Inc. (Cat. Nos. sc-39699). PD-L1 siRNA are described in Breton et al., J Clin Immunol. 2009 29(5): 637-45. Epub 2009 Jun. 27; Hobo et al., Blood, 2010, 116(22): 4501-4511.

In another embodiment, the above-mentioned PD-1 inhibitor is an agent that blocks the interaction between PD-1 and one or more signalling molecules involved in mediating the PD-1 inhibitory signal, such as SHP-1 and SHP-2. In an embodiment, the agent targets the immunoreceptor tyrosine-based inhibitory (ITIM) motif (residues 221 to 226) and/or the immunoreceptor tyrosine-based switch (ITSM) motif (residues 246 to 251) of PD-1, and blocks the recruitment of SHP-1 and/or SHP-2.

As noted above, latent HIV persists in a small fraction of resting memory CD4⁺ T cells in HAART-treated subjects. This HIV reservoir, which is not eliminated/purged by antiretroviral therapy, serves as a source of virus for reseeding the infection after HAART discontinuation. The results described herein demonstrate that PD-1 contributes to the inhibition of viral production in primary CD4⁺ T cells, and that blocking PD-1 stimulates/increases viral replication in these cells, and therefore that PD-1 blocking is useful for reactivating HIV replication in latently-infected cells, thus permitting elimination of HIV using antiretroviral drugs.

Accordingly, in another aspect, the present invention provides a method for reducing or eliminating a latent HIV reservoir in a cell comprising:

(a) performing the above-mentioned method for increasing or reactivating HIV replication in a cell (e.g., a latently HIV-infected cells); and

(b) contacting the cell with one or more antiretroviral agents.

In another aspect, the present invention provides a method for decreasing the number of latently HIV-infected cells in a subject, said method comprising administering to said subject an effective amount of:

(a) a PD-1 inhibitor; and

(b) one or more antiretroviral agents.

A PD-1 inhibitor may thus be co-administered (at the same time, or sequentially) with any antiretroviral drugs, such as antiretroviral drugs commonly used in HAART regimen. Typically, HAART usually involves a combination of (e.g., at least three) nucleoside reverse transcriptase inhibitors and frequently includes a protease inhibitor, or alternatively a non-nucleoside reverse transcriptase inhibitor. In an embodiment, the PD-1 inhibitor is administered prior to the antiretroviral agents. In another embodiment, the PD-1 inhibitor is administered to a patient already undergoing antiretroviral therapy.

### Pharmaceutical Compositions

In an embodiment, the composition of the present invention is a pharmaceutical composition and comprises a pharmaceutically acceptable carrier or excipient. As used herein “pharmaceutically acceptable carrier” or “excipient” includes any and all solvents, buffers, dispersion media, coatings, antibacterial and antifungal agents, isotonic and absorption delaying agents, and the like that are physiologically compatible. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art (Rowe et al., Handbook of pharmaceutical excipients, 2003, 4^{th} edition, Pharmaceutical Press, London UK). Except insofar as any conventional media or agent is incompatible with the active compound, use thereof in the pharmaceutical compositions of the invention is contemplated. The carrier can be suitable, for example, for intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration.

Therapeutic formulations may be in the form of liquid solutions or suspension; for oral administration, formulations may be in the form of tablets or capsules; and for intranasal formulations, in the form of powders, nasal drops, or aerosols.

Examples of formulations suitable for oral administration are (a) liquid solutions, such as an effective amount of active agent(s)/composition(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds/compositions of the invention include ethylenevinyl acetate copolymer particles, osmotic pumps, implantable infusion systems, and liposomes. Formulations for inhalation may contain excipients, (e.g., lactose) or may be aqueous solutions containing, for example, polyoxyethylene-9-lauryl ether, glycocholate and deoxycholate, or may be oily solutions for administration in the form of nasal drops, or as a gel.

For preparing pharmaceutical compositions from the compound(s)/composition(s) of the present invention, pharmaceutically acceptable carriers are either solid or liquid. Solid form preparations include powders, tablets, pills, capsules, cachets, suppositories, and dispersible granules. A solid carrier can be one or more substance, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired. The powders and tablets may typically contain from 5% or 10% to 70% of the active compound/composition. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use are prepared by dissolving the active compound(s)/composition(s) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Formulations to be used for in vivo administration are preferably sterile. This is readily accomplished, for example, by filtration through sterile filtration membranes.

The amount of the pharmaceutical composition (e.g., a PD-1 agonist, a PD-1 inhibitor) which is effective in the prevention and/or treatment of a particular disease, disorder or condition (e.g., HIV infection and/or HIV-related disease) will depend on the nature and severity of the disease, the chosen prophylactic/therapeutic regimen (i.e., compound, protein, cells), the target site of action, the patient's weight, special diets being followed by the patient, concurrent medications being used, the administration route and other factors that will be recognized by those skilled in the art. The dosage will be adapted by the clinician in accordance with conventional factors such as the extent of the disease and different parameters from the patient. Typically, 0.001 to 1000 mg/kg of body weight/day will be administered to the subject. In an embodiment, a daily dose range of about 0.01 mg/kg to about 500 mg/kg, in a further embodiment of about 0.1 mg/kg to about 200 mg/kg, in a further embodiment of about 1 mg/kg to about 100 mg/kg, in a further embodiment of about 10 mg/kg to about 50 mg/kg, may be used. The dose administered to a patient, in the context of the present invention should be sufficient to effect a beneficial prophylactic and/or therapeutic response in the patient over time. The size of the dose also will be determined by the existence, nature, and extent of any adverse side-effects that accompany the administration. Effective doses may be extrapolated from dose response curves derived from in vitro or animal model test systems. For example, in order to obtain an effective mg/kg dose for humans based on data generated from rat studies, the effective mg/kg dosage in rat may be divided by six.

In an embodiment, the above-mentioned treatment comprises the use/administration of more than one (i.e. a combination of) active/therapeutic agent (e.g., PD-1 agonists, PD-1 inhibitors). The combination of therapeutic agents and/or compositions of the present invention may be administered or co-administered (e.g., consecutively, simultaneously, at different times) in any conventional dosage form. Co-administration in the context of the present invention refers to the administration of more than one therapeutic in the course of a coordinated treatment to achieve an improved clinical outcome. Such co-administration may also be coextensive, that is, occurring during overlapping periods of time. For example, a first agent may be administered to a patient before, concomitantly, before and after, or after a second active agent is administered. The agents may in an embodiment be combined/formulated in a single composition and thus administered at the same time. In an embodiment, the one or more active agent(s) of the present invention is used/administered in combination with one or more agent(s) currently used to prevent or treat HIV infection and/or HIV-associated diseases, for example antiretroviral drugs including reverse transcriptase inhibitors (nucleoside and non-nucleoside) such as Efavirenz, Zidovudine (AZT), Lamivudine (3TC), Tenofovir and Emtricitabine, protease inhibitors such as Saquinavir, Ritonavir, Indinavir, Nelfinavir and Amprenavir, and integrase inhibitors such as Raltegravir. In an embodiment, the above-mentioned PD-1 agonist or PD-1 inhibitor is administered/used in combination with drugs commonly used in HAART regimens.

### Kits/Packages for the Treatment of HIV Infection

The invention further provides kits or packages comprising the above-mentioned agent (e.g., PD-1 agonist or PD-1 inhibitor) or composition together with instructions for its use for treating HIV infection or HIV/related diseases and/or for decreasing the number of latently HIV-infected cells in a subject. The kit may further comprise, for example, containers, buffers, a device (e.g., syringe) for administering the agent, or a composition comprising same, to a subject. The instruction may also comprise warnings of possible side effects and drug-drug or drug-food interactions.

### Screening Methods

The present invention also relates to methods for identifying agents that may be useful for modulating HIV replication based on PD-1 modulation.

In another aspect, the present invention provides a method for determining whether a test compound may be useful for modulating HIV replication, said method comprising:
(a) contacting a PD-1 or a functional variant or fragment thereof with said test compound;
(b) determining whether said test compound binds to said PD-1, functional variant or fragment thereof;
   wherein the binding of said test compound to said PD-1, functional variant or fragment thereof is indicative that said test compound may be useful for modulating HIV replication.

In an embodiment, the above-mentioned binding is determined by assessing whether said test compound inhibits or interferes with the binding of a PD-1 ligand (i.e., competes with said PD-1 ligand for binding to PD-1). In an embodiment, the above-mentioned PD-1 ligand is PD-L1 or PD-L2, or a variant or fragment thereof comprising a PD-1-binding domain.

In another embodiment, the above-mentioned method further comprises determining whether said test compound (which binds to PD-1) inhibits or increases PD-1 activity, for example using the method described below. An inhibition of PD-1 activity would be indicative that said test compound is a PD-1 inhibitor and thus may be used to stimulate HIV replication whereas an increase in PD-1 activity would be indicative that said test compound is a PD-1 agonist and thus may be used to inhibit HIV replication.

In another aspect, the present invention provides a method for determining whether a test compound may be useful for inhibiting HIV replication, said method comprising:
(a) contacting a cell expressing PD-1 or a functional variant or fragment thereof with said test compound;
(b) determining whether PD-1 activity and/or expression is increased in the presence of said test compound relative to the absence thereof;
   wherein an increase in said activity and/or expression in the presence of said of said test compound relative to the absence thereof is indicative that said test compound may be useful for inhibiting HIV replication.

In another aspect, the present invention provides a method for determining whether a test compound may be useful for increasing or stimulating HIV replication in a cell, said method comprising:
(a) contacting a cell expressing PD-1 or a functional variant or fragment thereof with said test compound;
(b) determining whether PD-1 activity and/or expression is decreased in the presence of said test compound relative to the absence thereof;
   wherein a decrease in said activity and/or expression in the presence of said of said test compound relative to the absence thereof is indicative that said test compound may be useful for increasing or stimulation HIV replication in a cell.

In another aspect, the present invention provides a method for determining whether a test compound may be useful (when used in combination with an antiretroviral agent) for decreasing the number of latently HIV-infected cells in a subject, said method comprising:
(a) contacting a cell expressing PD-1 or a functional variant or fragment thereof with said test compound;
(b) determining whether PD-1 activity and/or expression is decreased in the presence of said test compound relative to the absence thereof;
   wherein a decrease in said activity and/or expression in the presence of said of said test compound relative to the absence thereof is indicative that said test compound may be useful for decreasing the number of latently HIV-infected cells in a subject.

A homolog, variant and/or fragment of PD-1 which retains activity (i.e. a functional homolog, variant or fragment) may also be used in the uses and methods of the invention. Homologs include protein sequences, which are substantially identical to the amino acid sequence of a PD-1 (e.g., FIG. 8), sharing significant structural and functional homology with a PD-1. Variants include, but are not limited to, proteins or peptides, which differ from a PD-1 (e.g., FIG. 8) by any modifications, and/or amino acid substitutions, deletions or additions (e.g. fusion with another polypeptide). Modifications can occur anywhere including the polypeptide backbone, (i.e. the amino acid sequence), the amino acid side chains and the amino or carboxy termini. Such substitutions, deletions or additions may involve one or more amino acids. Fragments include a fragment or a portion of a PD-1 or a fragment or a portion of a homolog or variant of a PD-1 which retains PD-1 activity. As noted above, the domains and residues of human PD-1 and PD-L1 involved in their interaction is described in Lin et al., supra, and include the IgV domain of PD-1 (from about residues 35 to 145, and more particularly residues 64, 66, 68, 73-76, 78, 90, 122, 124, 126, 128, 130-132, 134 and 136). Based on this knowledge, the skilled person would be able to easily identify/prepare functionally active fragments and/or variants of PD-1, for example fragments and/or variants comprising the IgV domain of PD-1 or in which one or more (or all) of the above-mentioned residues are conserved, that could be used in the methods of the invention.

“Homology” and “homologous” and “homolog” refer to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “homologous” to or is a “homolog” of another sequence if the two sequences are substantially identical and the functional activity of the sequences is conserved (as used herein, the term ‘homologous’ does not infer evolutionary relatedness). Two nucleic acids or amino acid sequences are considered “substantially identical” if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%, e.g., with the sequences depicted in the instant Figures. As used herein, a given percentage of homology between sequences denotes the degree of sequence identity in optimally aligned sequences. An “unrelated” or “non-homologous” sequence shares less than 40% identity, though preferably less than about 25% identity, with the sequences depicted in the instant Figures.

Substantially complementary nucleic acids are nucleic acids in which the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, or at least 95%, e.g., with the sequences depicted in the instant Figures. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48: 443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001. An alternative indication that two nucleic acid sequences are substantially complementary is that the two sequences hybridize to each other under moderately stringent, or preferably stringent, more preferably highly stringent conditions. Hybridization to filter-bound sequences under moderately stringent conditions may, for example, be performed in 0.5 M NaHPO4, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO4, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York). Generally, stringent conditions are selected to be about 5° C. lower than the thermal melting point for the specific sequence at a defined ionic strength and pH.

The assay may in an embodiment be performed using an appropriate host cell comprising PD-1 activity. Such a host cell may be prepared by the introduction of a nucleic acid encoding PD-1 (e.g., comprising the nucleotide sequence set forth in FIG. 8B, or the coding sequence thereof, or a functional fragment/variant thereof having PD-1 activity) into the host cell and providing conditions for the expression of PD-1. Such host cells may be prokaryotic or eukaryotic, bacterial, yeast, amphibian or mammalian. In an embodiment, the above-mentioned nucleic acid encoding PD-1 is linked to transcriptional regulatory sequences, for example in an expression vector.

“Transcriptional regulatory sequence” or “transcriptional regulatory element” as used herein refers to DNA sequences, such as initiation and termination signals, enhancers, and promoters, splicing signals, polyadenylation signals which induce or control transcription of protein coding sequences with which they are operably linked. A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since enhancers generally function when separated from the promoters by several kilobases and intronic sequences may be of variable lengths, some polynucleotide elements may be operably-linked but not contiguous. As used herein, a transcriptional regulatory element “normally” associated with for example a PD-1 gene refers to such an element or a functional portion thereof derived from sequences operably-linked to for example a PD-1 gene in its naturally-occurring state (i.e., as it occurs in a genome in nature). In another embodiment, the construct may comprise an in frame fusion of a suitable reporter gene within the open reading frame of a PD-1 gene. The reporter gene may be chosen as such to facilitate the detection of its expression, e.g. by the detection of the activity of its gene product. Such a reporter construct may be introduced into a suitable system capable of exhibiting a change in the level of expression of the reporter gene in response to exposure a suitable biological sample. Such an assay would also be adaptable to a possible large scale, high-throughput, automated format, and would allow more convenient detection due to the presence of its reporter component.

PD-1 activity and/or expression may be measured using various methods well known in the art.

Expression levels may in general be detected by either detecting nucleic acids (e.g., mRNA) from the cells and/or detecting expression products (e.g., polypeptides or proteins).

Suitable methods or techniques for measuring/quantitating or detecting nucleic acids include, but are not limited to, polymerase chain reaction (PCR), reverse transcriptase-PCR (RT-PCR), in situ PCR, quantitative PCR (q-PCR), in situ hybridization, Southern blot, Northern blot, sequence analysis, microarray analysis, detection of a reporter gene, or other DNA/RNA hybridization platforms. The term “quantifying” or “quantitating” when used in the context of quantifying transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g., through generation of a standard curve). Alternatively, relative quantification can be accomplished by comparison of hybridization signals between two or more genes, or between two or more treatments to quantify the changes in hybridization intensity and, by implication, transcription level.

Methods to measure protein expression levels are well known in the art. Examples of such methods include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, flow cytometry, and assays based on a property of the protein including but not limited to DNA binding, ligand binding (e.g., binding to PD-L1 and/or PD-L2), or interaction with other protein partners.

PD-1 activity may be determined using methods well known in the art. For example, PD-1 activity may be determined by measuring the expression of one or more gene(s) (at the nucleic acid and/or polypeptide level) whose expression is modulated by PD-1 activity, such as CD55, NFKB2, FAM65A, DIP, STS-1, TPST2, E4F1, CST7, GNG4, CD70, BACH₂, REL, PAM, KIAA0831, LOC197322, IL2RA, IL13, LPIN1, CBFA2T3, KRT1, MT1A, ANKRD5, NQO1, KLF6, CENPE, SMOX, FBXO34, LZTS1, LAMP3, SPEN, SH2B3, TNF, BAT2D1, ZYX, SPTBN1, ATP1B1, SLA, PLAU, SOCS1, OSGIN1, BRD2, VGF, PTPN6, TNFSF14, IL2, CD97, RPL28, CSF2, CCAR1, RPL7L1, CD83, MIDN, BCL2L1, LUZP1, VHL, CCL20, PCNT, SPRY1, RUNX3, BCL2A1, MBP, RHOU, RDH10, HTR2B, DDEF1, GZMB, TJAP1, MACF1, RCBTB2, RGS16, JMJD1C, SPRY1, LTB, MYH9, CLIP3, GBE1, CCDC64, PHEX, SNX26, TAGAP, FAM50A, TRAF1, CDK5RAP2, TAF1C, KIAA1754, LRRC8C, SUPT6H, IL23A, SH2D2A, IL21R, ATP6V0A4, TNFRSF8, MAPRE2, TMEM158, ITGA5, JAM3, BAZ1A, 1L3, FOS, HES4, TIMP1, TNS3, NFKBIA, CGA, TSC22D1, ATP1B1, EIF4G3, ATP6V1B2, DUSP1, SLC9A1, MEF2D, SNAPC4, GPR171, CD27, ALDOC, TNFRSF21, DPP9, SRRM2, METT11D1, CD69, IRX5, TBC1D10C, KLF6, PLAGL2, KLF2, PRR14, BIRC3, FSCN1, IGFBP2, LTBP4, USP11, BHLHB2, ARC, PPP1R15A, AUTS2, RXRA, MARVELD3, ARG2, SETD2, CENPF, ADORA2A, FOSB, EGR2, LAIR2, CBX6, PHACTR4, CCL4L1, ULK1, PTPN22, GNL3L, ZCCHC6, PRKCH, MFSD2, BIRC3, TMEM187, C6orf190, ITPR3, ADM, MT2A, EOMES, POU2AF1, NFATC1, C1orf165, ZFP36, BCL9, NOTCH1, POLE, LY96, CREBBP, EGR4, ACVR1, PFKFB4, NR4A2, MYC, CCL1, CXCR3, ICOS, MAGI and/or FXYD5, as disclosed in PCT publication No. WO 09/067,812. For example, PD-1 engagement has been shown to be associated with decreased IL-2 levels. Therefore, the effect on a test compound on PD-1 activity may be determined by measuring the levels of IL-2 mRNA or polypeptide in PD-1-expressing cells in the presence and absence of the test compound. A decrease in IL-2 levels in the presence of the compound would be indicative that the compound is a PD-1 agonist (and thus may be useful for inhibiting HIV replication), whereas an increase in IL-2 levels in the presence of the compound would be indicative that the compound is a PD-1 inhibitor (and thus may be useful for reactivating HIV replication in latently HIV-infected cells).

Also, given the known effect of PD-1 engagement on cell proliferation (e.g., T cell proliferation), the effect on a test compound on PD-1 activity may be determined by measuring the proliferation of the PD-1-expressing cells (using well known methods such as ³H-thymidine incorporation or CFSE dilution) in the presence and absence of the test compound. A decrease in proliferation in the presence of the compound would be indicative that the compound is a PD-1 agonist (and thus may be useful for inhibiting HIV replication), whereas an increase in proliferation in the presence of the compound would be indicative that the compound is a PD-1 inhibitor (and thus may be useful for reactivating HIV replication in latently HIV-infected cells).

In an embodiment, the above-mentioned PD-1-expressing cell endogenously expresses PD-1. In another embodiment, the above-mentioned PD-1-expressing cell recombinantly expresses PD-1 (i.e., has been transfected or transformed with a nucleic acid encoding PD-1, or has been genetically modified to induce the expression/overexpression of endogenous PD-1). In another embodiment, the above-mentioned PD-1-expressing cell is a T cell, in a further embodiment a CD4⁺ T cell.

Screening assay systems may comprise a variety of means to enable and optimize useful assay conditions. Such means may include but are not limited to: suitable buffer solutions, for example, for the control of pH and ionic strength and to provide any necessary components for optimal activity and stability (e.g., protease inhibitors), temperature control means for optimal activity and/or stability, of PD-1, and detection means to enable the detection of its activity. A variety of such detection means may be used, including but not limited to one or a combination of the following: radiolabelling, antibody-based detection, fluorescence, chemiluminescence, spectroscopic methods (e.g., generation of a product with altered spectroscopic properties), various reporter enzymes or proteins (e.g., horseradish peroxidase, green fluorescent protein), specific binding reagents (e.g., biotin/(strept)avidin), and others.

The screening methods mentioned herein may be employed either with a single test compound or a plurality or library (e.g., a combinatorial library) of test compounds. In the latter case, synergistic effects provided by combinations of compounds may also be identified and characterized. In certain embodiments, one or a plurality of the steps of the screening/testing methods of the invention may be automated.

### Enrichment of Latently HIV-Infected Cells

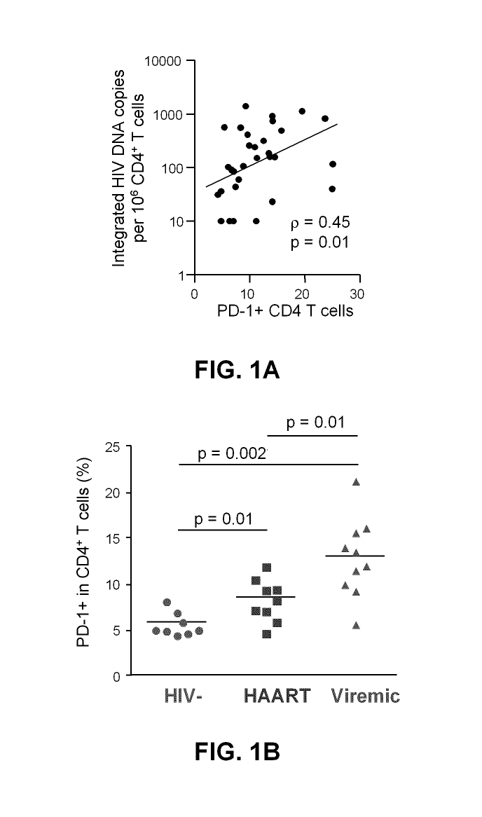

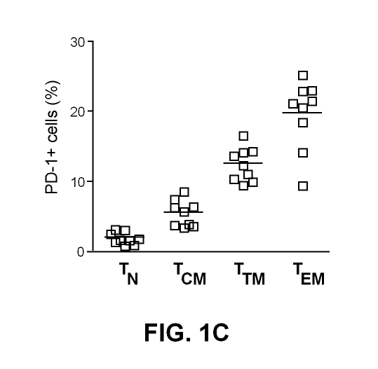

The data presented herein indicates that PD-1 expressing cells are more likely to harbour integrated HIV DNA, a hallmark of latently HIV-infected cells. Accordingly, in another aspect, the present invention provides a method for enriching a cell population in latently HIV-infected cells, the method comprising contacting said cell population with an agent binding to PD-1; and isolating/purifying the cells binding to the ligand. The agent may be any molecule capable of specifically binding to PD-1, such as antibodies, a PD-1 ligand (PD-L1 or a PD-1 binding fragment thereof). In an embodiment, the agent is conjugated to a label, such as a fluorescent label, that permits the detection and purification of cells on which the agent is bound using commonly used techniques (e.g., fluorescent activated cell sorting (FACS) or any other affinity-based cell enrichment technique). In an embodiment, the bound agent may be indirectly detected, for example using a second agent that specifically recognizes the first agent (e.g., a secondary antibody). Such second agent is typically labelled to allow the detection of the complex. In another embodiment, the method further comprise contacting the cell population with one or more markers. For example, FIG. 1C shows that the effector memory cell population (CD45RA⁻ CCR7⁺ CD27⁻, T_{EM}) contains a higher proportion of PD-1 expressing cell as compared to other cell subsets naïve (CD45RA⁺ CCR7⁺ CD27⁺, T_{N}), central memory (CD45RA⁻ CCR7⁺ CD27⁺, T_{CM}) and transitional memory (CD45RA⁻ CCR7⁻ CD27⁺, T_{TM}). Therefore, the above-mentioned may further comprises contacting the cell with an agent that binds to CD45RA, CCR7 and/or CD27.

### MODE(S) FOR CARRYING OUT THE INVENTION

The present invention is illustrated in further details by the following non-limiting examples.

### Example 1

### Materials and Methods

Human Subjects.

Ten HIV-chronically infected subjects enrolled in this study and signed informed consent approved by the Royal Victoria Hospital and the CR-CHUM hospital review board. None of these subjects received antiretroviral therapy at the time of study. Plasma viremia were measured by the Amplicor™ HIV-1 monitor ultrasensitive Method (Roche). All subjects underwent leukapheresis to collect large numbers of PBMCs.

Stimulation of CD4⁺ T Cells.

PBMCs from HIV-infected donors were isolated from whole blood by density gradient centrifugation (Ficoll) and resuspended in RPMI supplemented with 10% Fetal Bovine Serum (FBS). CD4⁺ T cells were isolated by negative selection on a Robosep™ (Stemcell Technologies—EasySep™ Human CD4⁺ T cell enrichment kit, Cat. No. 19052). Purified CD4⁺ T cells (more than 90% pure, as determined by flow cytometry) were distributed at 1×10⁶ cells/ml in 48 well plates in 1 ml of RPMI supplemented with 10% FBS and Penicillin (100 U/ml)+Streptomycin (100 μg/ml). Purified mouse anti-CD3 (BD BioSciences, Cat. No. 555330), purified mouse anti-CD28 (BD BioSciences, Cat. No. 555726), murine IgG2a human PD-L1 chimera (Freeman, G. J. et al. (2000) J. Exp. Med. 192:1027, commercially available from R&D Systems, Catalog Number: 156B7) or isotype control IgG2a (Sigma, Cat. No. M5409.1MG) were covalently attached to superparamagnetic polystyrene beads (4.5 μm diameter) coated with a monoclonal human anti-mouse IgG antibody via a DNA linker (CELLection™ Pan Mouse IgG Dynabeads™ (Invitrogen, Cat. No. 115.31D). To prepare 2×10⁷ beads, 140 ng of anti-CD3 antibody, 33 ng of anti-CD28 and 500 ng of Human PD-L1-murine IgG2a chimera (or isotype control) were used. Cells were stimulated with beads at a ratio of 1:2.

HIV-1 Released Virus Quantification.

After 24 h of stimulation, 500 μl of supernatant was harvested and replaced with 500 μl of fresh medium. Viral particles were pelleted by centrifugation for 60 min at 17,000 rpm at 4° C. To generate the standard curve, a sample of titer-known HIV-1 was pelleted in the same run. Viral pellets were used to extract the viral RNA using the QIAamp™ viral RNA mini kit (Qiagen, Cat. No. 52906). The purified RNA was then used as a matrix for a two-step quantitative real-time reverse transcription-PCR(RT-PCR followed by qRT-PCR). For each sample, a minimum of 2 independent replicates (separate wells) were performed, including the ACH2 RNA sample as a standard ranging from 300000 copies to 3 copies. Total viral RNA (17 μl) was first treated with 1 U of DNase in DNase I reaction buffer 1× for 10 min at 25° C. The DNase was inactivated with 1 μL of 25 mM EDTA for 10 min at 65° C. Total viral RNA was then reverse-transcribed into cDNA for quantitative PCR analysis. RT-PCR was performed in 50 μl of solution containing 22 μl of DNase treated RNA, 0.5 μl of each Gag gene-specific primers (50 μM each), LM667 (5′-ATG CCA CGT AAG CGA AAC TCT GGC TAA CTA GGG AAC CCA CTG-3′, SEQ ID NO: 5) and GagR (5′-AGC TCC CTG CTT GCC CAT A-3′, SEQ ID NO: 6), 2 μl of Superscript™ III RT/Platinum™ Taq mix and 1× Reaction mix (Superscript™ One-Step RT-PCR kit, Invitrogen, Cat. No. 10928-042) in a final volume of 50 μl. No-template samples were used as negative controls. The running conditions were as follows: reverse transcription 30 min at 50° C., denaturation 2 min at 94° C. followed by 20 cycles at 94° C. for 15 sec (denaturation), 62° C. for 30 s (annealing), and 68° C. for 1 min (extension). The reaction was achieved by a final elongation at 68° C. for 5 min before cooling gradually to 4° C. The cDNAs were diluted 10-fold with DNase-RNase free water, then subjected to quantitative real-time PCR analysis. Quantitative Real-Time PCR (qRT-PCR) experiments were performed with a LightCycler™ Carousel-based system (Roche). Water was included as a no-template control. All reactions were carried out in 20 μl reaction mixtures containing 6.4 μl of cDNAs, 0.3 μl of Taq DNA polymerase (Invitrogen), 1× Jumpstart™ mix (Sigma), 1.8 μl MgCl₂ 25 mM, 0.25 μl of each Gag gene-specific primers (100 μM each), Lambda T (5′-ATG CCA CGT AAG CGA AAC T-3′, SEQ ID NO: 7) and A55M (5′-GCT AGA GAT TTT CCA CAC TGA CTA A-3′, SEQ ID NO: 8), 0.5 μl each hybridization probes (8 μM each) LTR-LC (LCred640-5′-CAC TCA AGG CAA GCT TTA TTG AGG C-3′-Phosphate, SEQ ID NO: 9) and LTR-FL (5′-CAC AAC AGA CGG GCA CAC ACT ACT TGA-3′-Fluorescein, SEQ ID NO: 10). The running conditions were as follows: 4 min at 95° C., followed by 50 cycles of 95° C. for 10 sec (denaturation), 60° C. for 10 s (annealing), and 72° C. for 9 s (extension). Following the PCR reaction, melting curve analysis was performed to control amplification specificity by measuring the fluorescence intensity across the temperature interval from 45° C. to 95° C. The absence of nonspecific products or primer dimers was indicated by observation of a single melting peak in melting curve analysis.

Cell supernatants were also collected after 3, 6 and 9 days of stimulation, and p24 levels were measured by an in-house sandwich ELISA using the monoclonal antibody 183-H12-5C (coating) and the biotinylated antibody 31.90.25, two antibodies recognizing different epitopes of the HIV-1 major viral core protein p24. Briefly, flat-bottom 96-well plates (Immulon 2; Dynatech, Ltd.) were initially coated with 183-H12-5C, a monoclonal anti-p24 antibody. After the wells were washed and blocked with 1% bovine serum albumin (Sigma, St. Louis, Mo.), viral lysates were added to the wells at various dilutions, along with samples of known p24 concentration, in order to establish a standard curve. After a 60-min incubation at 37° C., the plates were washed, and a second biotinylated anti-p24 monoclonal antibody (clone 31-90-25) was then added. After a 45 min. incubation at 37° C., the plates were washed, and a spreptavidin-peroxidase conjugate (Steptavidin-HRP-40; Research Diagnostics) was added; this was followed by the addition of the TMB-S substrate (Cedarlane, Inc.). After 30 min at room temperature, the reaction was terminated by adding 1 M H₃PO₄, and the absorbance was measured at 450 nm. Unknown p24 values were calculated on the basis of regression analysis of p24 standards over a linear range of 2.5 to 160 pg/ml.

### Example 2

### PD-1 Expression in HIV-Infected Subjects

The results depicted in FIG. 1A show that there is a correlation between the frequency of CD4⁺ T cells expressing PD-1 and the frequency of CD4⁺ T cells harbouring integrated HIV DNA in HIV-infected subjects, suggesting that PD-1 expressing cells are more likely to harbour integrated HIV DNA. FIG. 1B demonstrates that the frequency of cells expressing PD-1 is increased during HIV infection, and cannot be normalized by HAART. The frequency of PD-1 expressing cells in various CD4 T cells subsets, namely naïve (CD45RA⁺ CCR7⁺ CD27⁺, T_{N}), central memory (CD45RA⁻ CCR7⁺ CD27⁺, T_{CM}), transitional memory (CD45RA⁻ CCR7⁺ CD27⁺, T_{TM}) and effector memory (CD45RA⁻ CCR7⁺ CD27⁻, T_{EM}), from 9 virally suppressed subjects is shown in FIG. 1C, with T_{EM}>T_{TM}>T_{CM}>T_{N}.

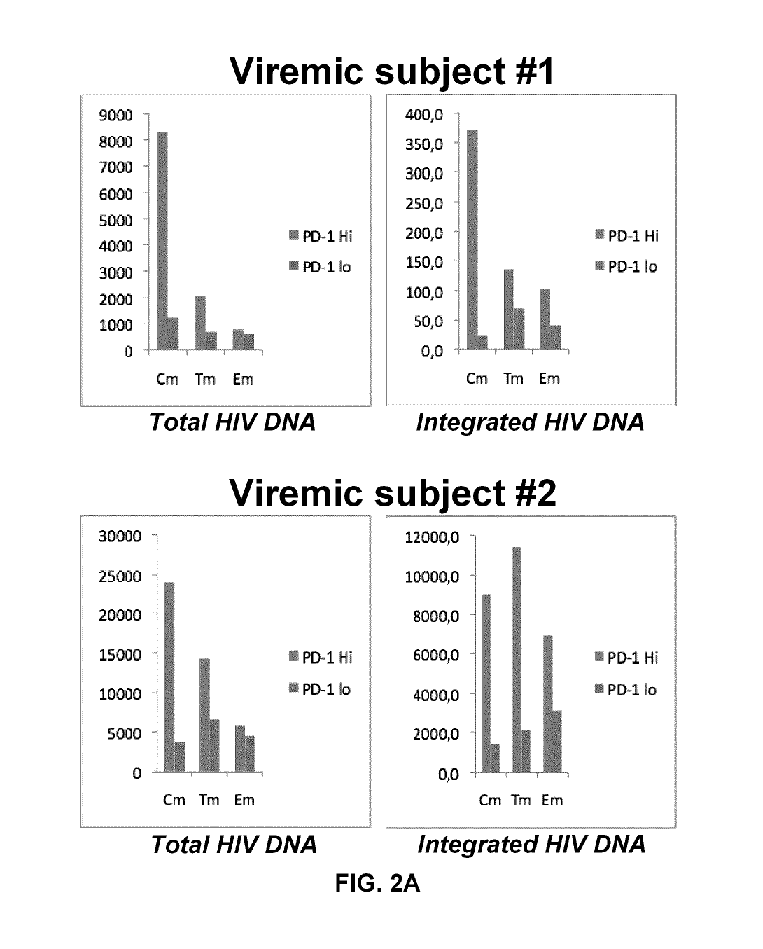

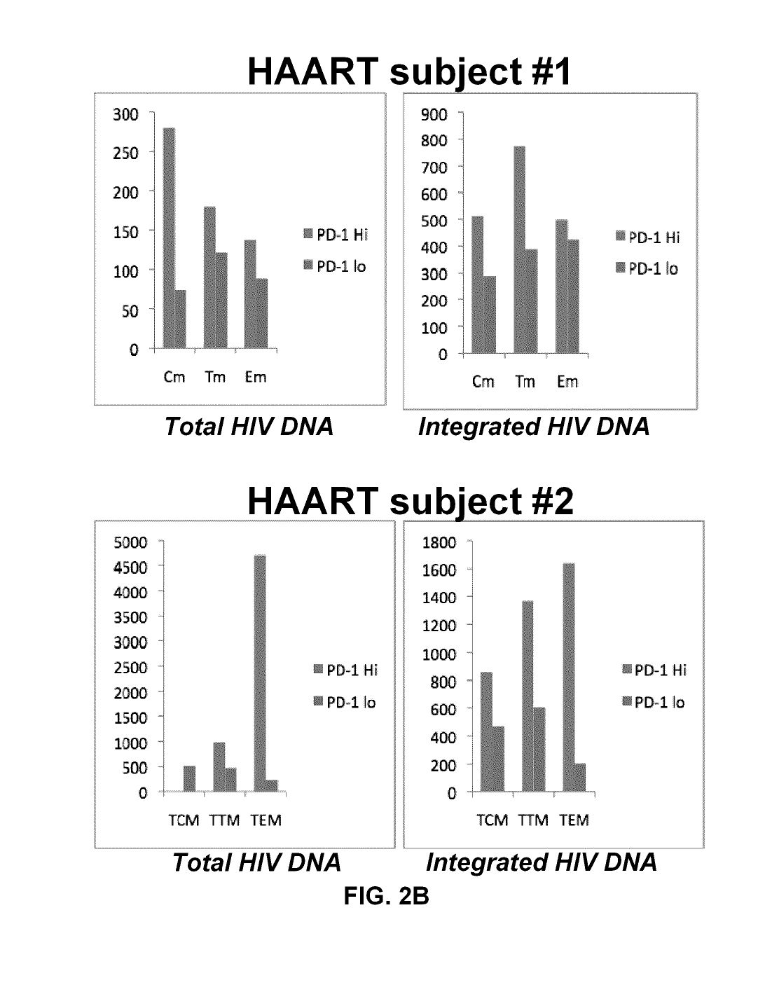

The frequency of PD-1^{hi} and PD-1^{lo} cells harbouring HIV DNA and integrated HIV DNA in untreated HIV infected subjects and virally suppressed subjects is depicted in FIGS. 2A and 2B, respectively. The results shows that PD-1^{hi} cells are enriched in total and integrated HIV DNA when compared to PD-1^{lo} cells in all memory CD4 T cell subsets, suggesting that PD-1^{hi} cells constitute a preferential reservoir for the virus.

### Example 3

### PD-1 Triggering Inhibits HIV Replication in Primary CD4⁺ T Cells

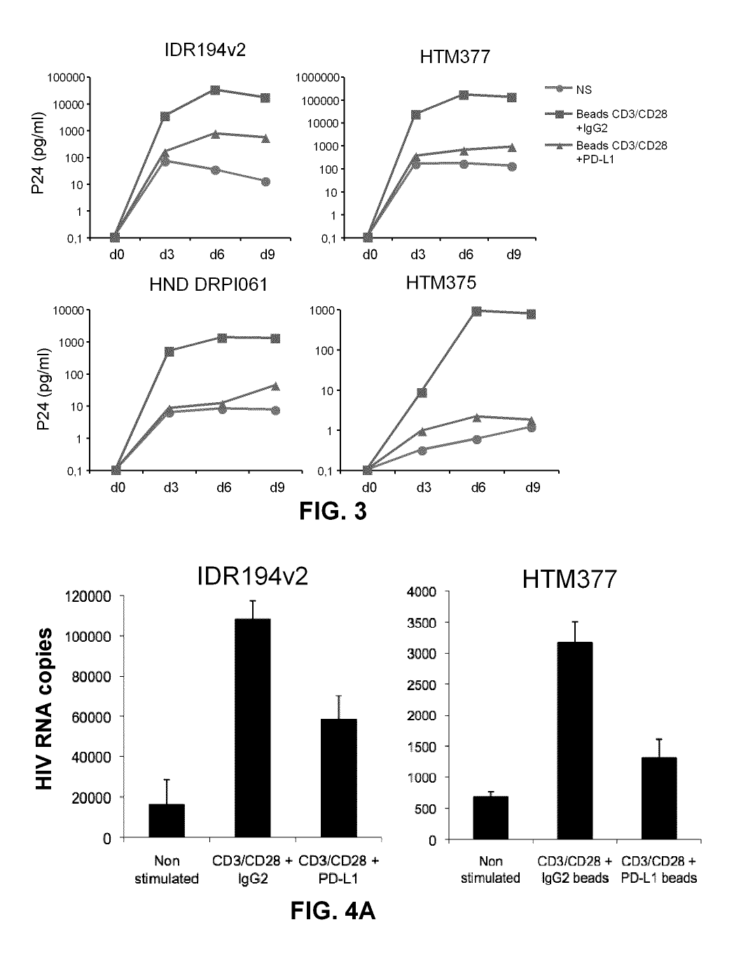

The effect of PD-1 triggering on HIV replication was assessed in primary CD4⁺ T cells purified from 6 viremic donors (results from 4 donors are illustrated in FIG. 3A). CD4⁺ T cells were isolated by negative selection and stimulated with anti-CD3+anti-CD28 antibodies with or without co-triggering of PD-1 by the murine IgG2a human PD-L1 chimera. PD-1 triggering inhibited HIV replication in primary CD4⁺ T cells after 3, 6 and 9 days of stimulation (mean percentages of inhibition with PD-L1 relative to isotype control=95.3, 99.0 and 98.2% after 3, 6 and 9 days, respectively).

### Example 4

### PD-1 Triggering Inhibits Early HIV Production in Primary CD4⁺ T Cells

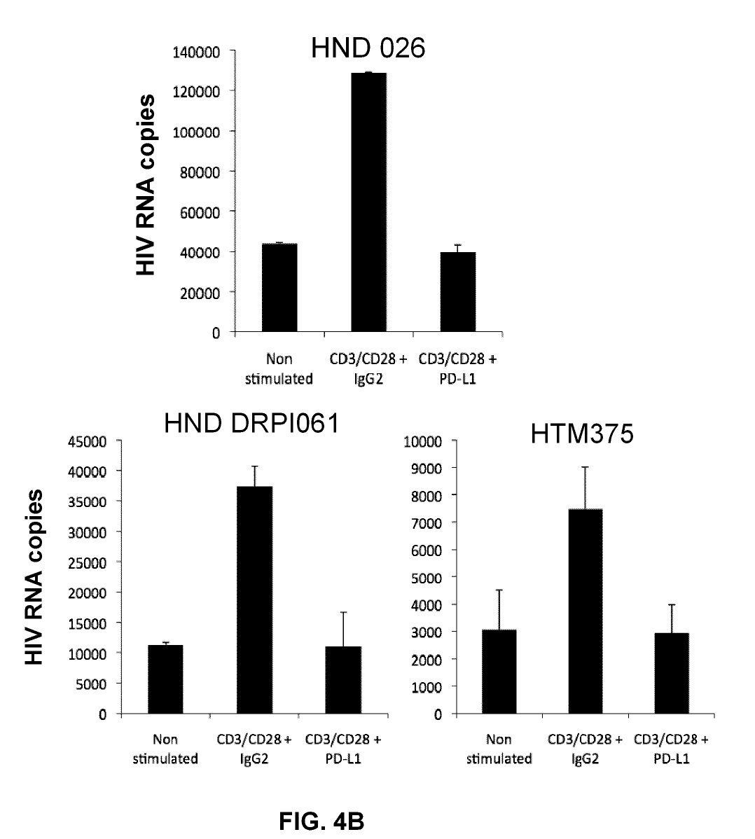

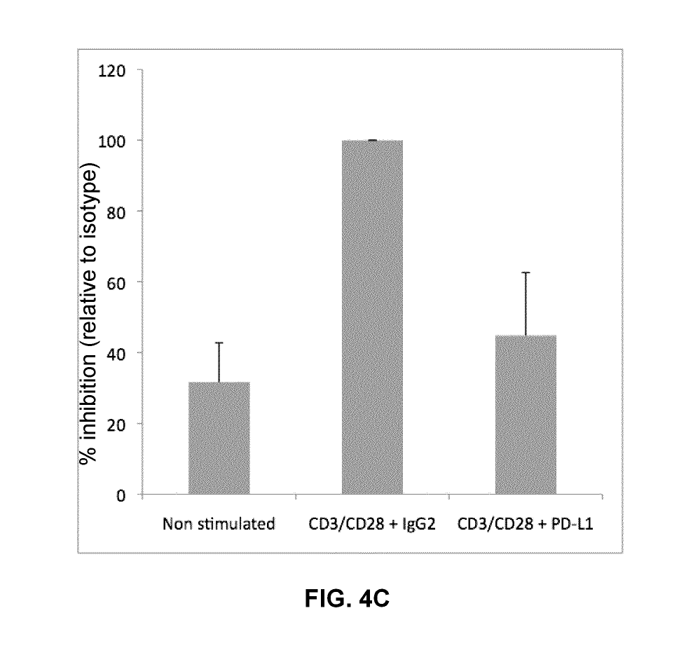

Since PD-1 is a negative regulator of T cell activation, one may hypothesize that the inhibition of HIV replication observed in Example 3 could be attributed to the limited activation levels of bystander CD4⁺ T cells, thereby limiting the number of new target cells available for de novo infections. To rule out this possibility, the above experiments were repeated, and early HIV production was determined by ultrasensitive RT-PCR after 24 hours of stimulation. The results depicted at FIGS. 4A and 4B indicate that early HIV production was inhibited after PD-1 engagement in the 5 donors tested, indicating that PD-1 triggering directly impacts on HIV production/replication. FIG. 4C depicts the percentage of inhibition of PD-1 engagement (relative to isotype controls) obtained in 7 donors.

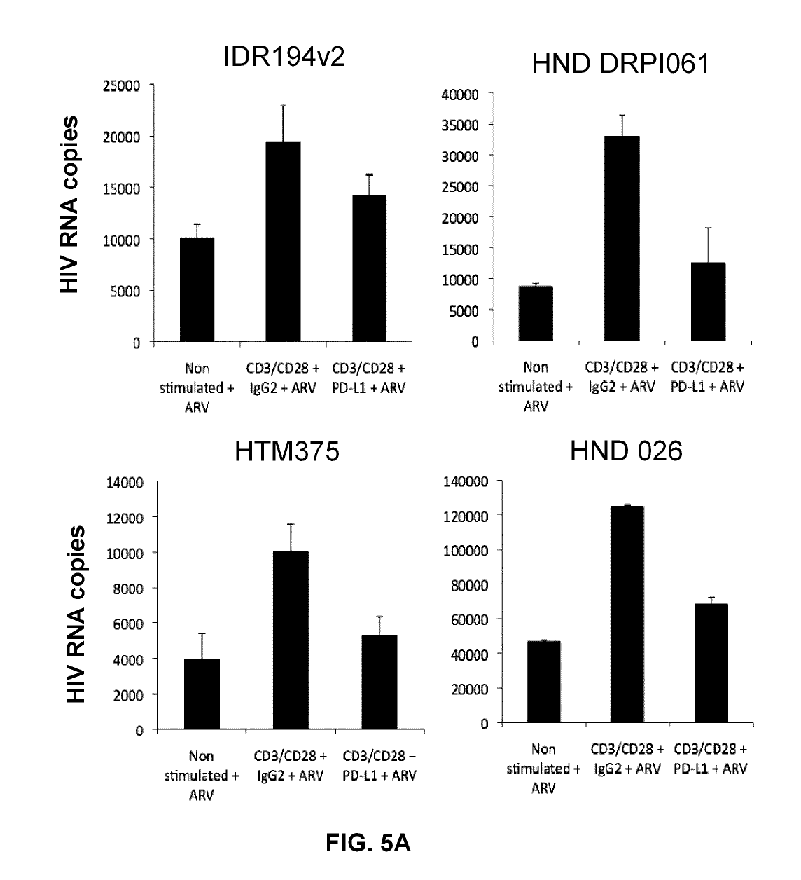

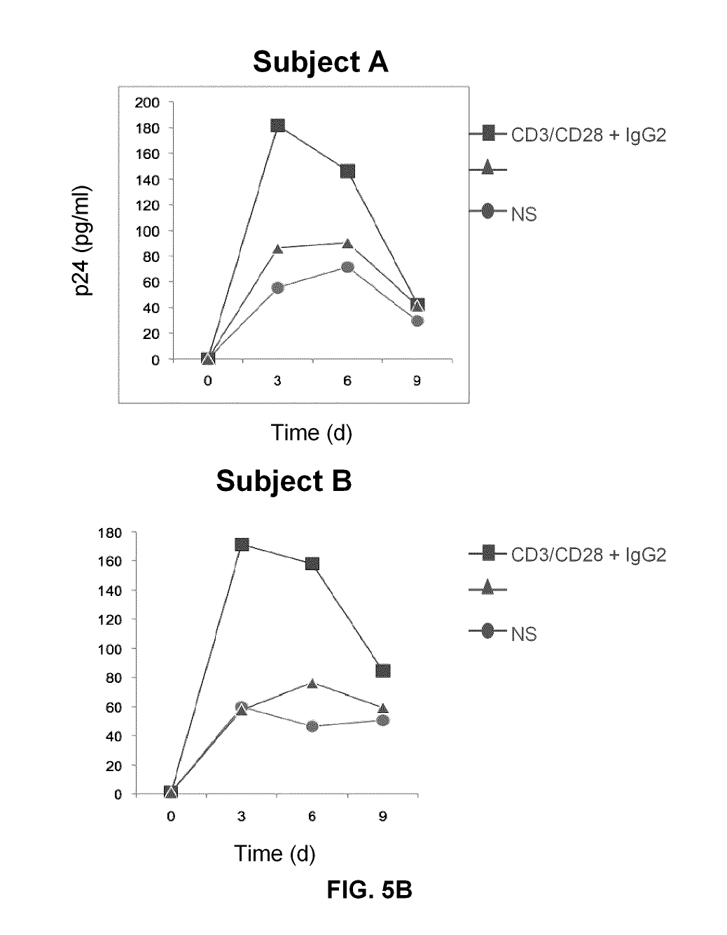

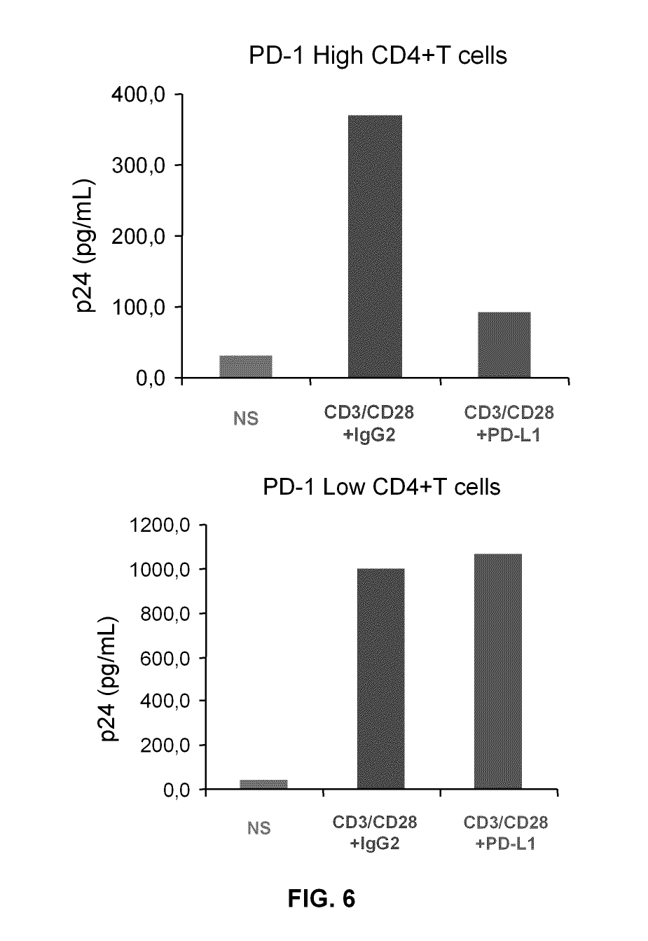

In order to confirm this result, the same experiment was repeated but in the presence of antiretroviral molecules (2 μM zidovudine (AZT), 2 μM Lamivudine (3TC), and 200 nM Saquinavir or Ritonavir, obtained through the AIDS Reagent program), thus allowing the assessment of the role of PD-1 engagement in a single round infection system. In accordance with the observations described above, HIV production (FIG. 5A) and early HIV production (FIG. 5B) was inhibited after engagement of PD-1 with its ligand in the presence of antiretroviral molecules. FIG. 6 shows that the effect of PD-1 triggering on HIV replication occurs only in primary CD4⁺ T cells expressing high levels of PD-1, confirming the role of the PD-1 pathway in the control of HIV replication.

Altogether, these results indicate that PD-1 engagement by an agonist (i.e. PD-L1, a natural PD-1 ligand) interaction directly inhibits HIV production in primary CD4⁺ T cells from viremic donors, and thus that PD-1 triggering could contribute to the establishment and maintenance of viral latency in CD4⁺ T cells.

### Example 5

### Disruption of the PD-1/PD-L1 Interaction Induces Viral Production in Primary

CD4⁺ T Cells

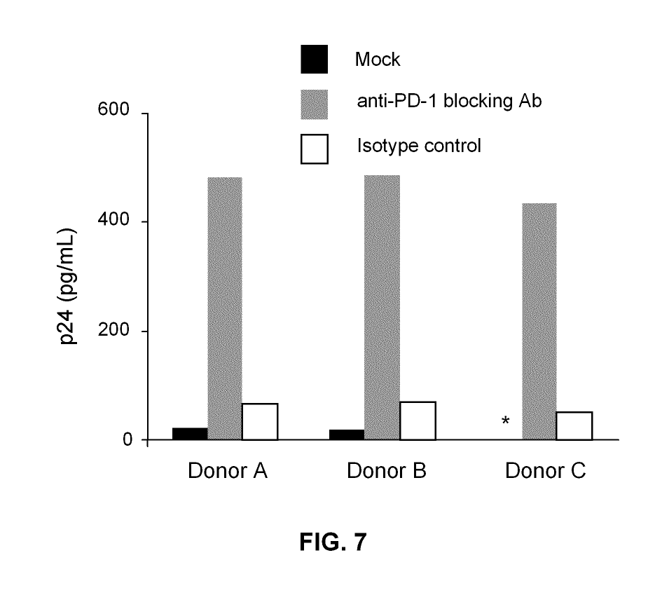

The data presented above shows that the triggering of the PD-1 pathway inhibits HIV production by infected CD4⁺ T cells. The effect of an antibody blocking the PD-1/PD-L1 interaction on viral production in CD4⁺ T cells was evaluated (FIG. 7). CD4⁺ T cells from viremic donors were isolated by negative magnetic selection as described above, and incubated with an anti-PD-1 antibody that prevents the interaction of PD-1 with its natural ligand PD-L1. After 3 days of culture, it was observed that blocking PD-1/PD-L1 interaction enhances the spontaneous release of HIV-1 virions by CD4⁺ T cells from 3 donors. This observation indicates that the PD-1/PD-L1 interaction contributes to the inhibition of viral production in primary CD4⁺ T cells, and thus that PD-1 inhibition could be used to reactivate HIV production in latently HIV-infected CD4⁺ T cells in virally suppressed subjects.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

1. A method for inhibiting Human Immunodeficiency Virus (HIV) replication in a cell; the method comprising: contacting the cell with a Programmed Death-1 (PD-1) agonist.

2. The method of claim 1, wherein the cell is a CD4+ T cell.

3. The method of claim 1, wherein said agonist comprises a PD-1 ligand selected from the group consisting of a Programmed Death Ligand-1 polypeptide, or an extracellular domain thereof.

4-13. (canceled)

14. A method for increasing HIV replication in a CD4⁺ T cell in vitro or in vivo, comprising contacting the CD4⁺ T cell with a PD-1 inhibitor, wherein the CD4⁺ T cell is an HIV-infected or a latently HIV-infected cell.

15. (canceled)

16. (canceled)

17. The method of claim 14, wherein the PD-1 inhibitor is an anti-PD-1 antibody or antigen-binding fragment thereof.

18. (canceled)

19. (canceled)

20. A method for reducing or eliminating a latent HIV reservoir in a CD4+ T cell in vitro or in vivo, comprising:
(a) contacting the CD4+ T cell with a PD-1 inhibitor or PD-1 agonist; and
(b) contacting the CD4+ T cell with one or more antiretroviral agents.

21-47. (canceled)

48. A composition for inhibiting HIV replication in a cell in vitro or in vivo, said composition comprising a PD-1 agonist and a carrier.

49. (canceled)

50. A composition for increasing HIV replication in a cell in vitro or in vivo, wherein the composition comprises an effective amount of a PD-1 inhibitor and a carrier.

51. A composition for reducing or eliminating a latent HIV reservoir in a cell in vitro or in vivo, the composition comprising a PD-1 inhibitor, one or more antiretroviral agents, and a pharmaceutically acceptable carrier.

52-56. (canceled)

57. The composition of claim 51 wherein the PD-1 inhibitor and the antiretroviral agents are formulated to be administered as a single composition.

58. The composition of claim 51 wherein the PD-1 inhibitor and the antiretroviral agents are formulated to be administered in two or more compositions.


SEQUENCE LISTING (19 sequences)

SEQ ID NO: 1 - DNA, 2115 bp, Homo sapiens
agtttcccttccgctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaaggaaaa

SEQ ID NO: 2 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 3 - DNA, 1553 bp, Homo sapiens
cgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaaaaaaaaaaaaaa

SEQ ID NO: 4 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 5 - DNA, 42 bp, Artificial Sequence
  Description of Artificial Sequence Synthetic primer
atgccacgtaagcgaaactctggctaactagggaacccactg

SEQ ID NO: 6 - DNA, 19 bp, Artificial Sequence
  Description of Artificial Sequence Synthetic primer
agctccctgcttgcccata

SEQ ID NO: 7 - DNA, 19 bp, Artificial Sequence
  Description of Artificial Sequence Synthetic primer
atgccacgtaagcgaaact

SEQ ID NO: 8 - DNA, 25 bp, Artificial Sequence
  Description of Artificial Sequence Synthetic primer
gctagagattttccacactgactaa

SEQ ID NO: 9 - DNA, 25 bp, Artificial Sequence
  Description of Artificial Sequence Synthetic probe
cactcaaggcaagctttattgaggc

SEQ ID NO: 10 - DNA, 27 bp, Artificial Sequence
  Description of Artificial Sequence Synthetic probe
cacaacagacgggcacacactacttga

SEQ ID NO: 11 - protein, 7 aa, Artificial Sequence
  Description of Artificial Sequence Synthetic peptide
DIEGRMD

SEQ ID NO: 12 - protein, 6 aa, Artificial Sequence
  Description of Artificial Sequence Synthetic peptide
IEGRMD

SEQ ID NO: 13 - DNA, 2418 bp, Homo sapiens
gcaaaccttaagctgaatgaacaacttttcttctcttgaatatatcttaacgccaaattttgagtgcttttttgttacccatcctcatatgtcccagctagaaagaatcctgggttggagctactgcatgttgattgttttgtttttccttttggctgttcattttggtggctactataaggaaatctaacacaaacagcaactgttttttgttgtttacttttgcatctttacttgtggagctgtggcaagtcctcatatcaaatacagaacatgatcttcctcctgctaatgttgagcctggaattgcagcttcaccagatagcagctttattcacagtgacagtccctaaggaactgtacataatagagcatggcagcaatgtgaccctggaatgcaactttgacactggaagtcatgtgaaccttggagcaataacagccagtttgcaaaaggtggaaaatgatacatccccacaccgtgaaagagccactttgctggaggagcagctgcccctagggaaggcctcgttccacatacctcaagtccaagtgagggacgaaggacagtaccaatgcataatcatctatggggtcgcctgggactacaagtacctgactctgaaagtcaaagcttcctacaggaaaataaacactcacatcctaaaggttccagaaacagatgaggtagagctcacctgccaggctacaggttatcctctggcagaagtatcctggccaaacgtcagcgttcctgccaacaccagccactccaggacccctgaaggcctctaccaggtcaccagtgttctgcgcctaaagccaccccctggcagaaacttcagctgtgtgttctggaatactcacgtgagggaacttactttggccagcattgaccttcaaagtcagatggaacccaggacccatccaacttggctgcttcacattttcatccccttctgcatcattgctttcattttcatagccacagtgatagccctaagaaaacaactctgtcaaaagctgtattcttcaaaagacacaacaaaaagacctgtcaccacaacaaagagggaagtgaacagtgctatctgaacctgtggtcttgggagccagggtgacctgatatgacatctaaagaagcttctggactctgaacaagaattcggtggcctgcagagcttgccatttgcacttttcaaatgcctttggatgacccagcactttaatctgaaacctgcaacaagactagccaacacctggccatgaaacttgccccttcactgatctggactcacctctggagcctatggctttaagcaagcactactgcactttacagaattaccccactggatcctggacccacagaattccttcaggatccttcttgctgccagactgaaagcaaaaggaattatttcccctcaagttttctaagtgatttccaaaagcagaggtgtgtggaaatttccagtaacagaaacagatgggttgccaatagagttattttttatctatagcttcctctgggtactagaagaggctattgagactatgagctcacagacagggcttcgcacaaactcaaatcataattgacatgttttatggattactggaatcttgatagcataatgaagttgttctaattaacagagagcatttaaatatacactaagtgcacaaattgtggagtaaagtcatcaagctctgtttttgaggtctaagtcacaaagcatttgttttaacctgtaatggcaccatgtttaatggtggttttttttttgaactacatctttcctttaaaaattattggtttctttttatttgtttttaccttagaaatcaattatatacagtcaaaaatatttgatatgctcatacgttgtatctgcagcaatttcagataagtagctaaaatggccaaagccccaaactaagcctccttttctggccctcaatatgactttaaatttgacttttcagtgcctcagtttgcacatctgtaatacagcaatgctaagtagtcaaggcctttgataattggcactatggaaatcctgcaagatcccactacatatgtgtggagcagaagggtaactcggctacagtaacagcttaattttgttaaatttgttctttatactggagccatgaagctcagagcattagctgacccttgaactattcaaatgggcacattagctagtataacagacttacataggtgggcctaaagcaagctccttaactgagcaaaatttggggcttatgagaatgaaagggtgtgaaattgactaacagacaaatcatacatctcagtttctcaattctcatgtaaatcagagaatgcctttaaagaataaaactcaattgttattcttcaacgttctttatatattctacttttgggta

SEQ ID NO: 14 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 15 - protein, 247 aa, Mus sp.
MLLLLPILNLSLQLHPVAALFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTWPLHVFIPACTIALIFLAIVIIQRKRI

SEQ ID NO: 16 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPSCIIAFIFIATVIALRKQLCQKLYSSKDTTKRPVTTTKREVNSAI

SEQ ID NO: 17 - protein, 290 aa, Mus sp.
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 18 - protein, 137 aa, Homo sapiens
PPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 19 - protein, 137 aa, Mus sp.
SLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMV